(19) United States
(12) Patent Application Publication
(10) Pub. No.: US 2025/0034510 A1
(43) Pub. Date: Jan. 30, 2025

(54) SYSTEMS, DEVICES, AND METHODS FOR BIOLOGICAL MATERIAL RESTORATION AND/OR CRYOPRESERVATION

(71) Applicant: Oregon State University, Corvallis, OR (US)

(72) Inventors: Morgan Giers, Corvallis, OR (US); Ward Shalash, Albany, OR (US); Adam Higgins, Corvallis, OR (US)

(73) Assignee: Oregon State University, Corvallis, OR (US)

(21) Appl. No.: 18/228,557

(22) Filed: Jul. 31, 2023

Related U.S. Application Data
(60) Provisional application No. 63/515,314, filed on Jul. 24, 2023.

(51) Int. Cl. C12M 1/00; A01N 1/02; C12M 1/33

(52) U.S. Cl. CPC C12M 45/06; A01N 1/0205; C12M 45/02

(57) ABSTRACT

Disclosed herein is a loading apparatus for restoration and/or cryopreservation of biological materials, a system comprising the same, as well as a method for using the loading apparatus and/or system to restore and/or cryopreserve biological materials. The disclosed loading apparatus and method for using the same focuses on utilizing cycles of applying and removing a load (e.g., tension and/or compression pressure) applied to the biological material that facilitate free-swelling of the biological material back to its natural state without the applied load, wherein the free-swelling promotes dispersion of a cryopreservation agent, therapeutic agent, restoration agent, or a combination thereof throughout the surface area of the biological material.

### CROSS REFERENCE TO RELATED APPLICATION

This application claims the benefit of the earlier filing date of U.S. Provisional Application No. 63/515,314, filed Jul. 24, 2023, which is incorporated by reference herein in its entirety.

### FIELD

This disclosure relates to embodiments of an apparatus, system, and method for cryopreserving and/or restoring biological materials.

### BACKGROUND

Restoration and cryopreservation of biological materials like tissues is a critical technique in the fields of medicine, research, and regenerative therapies, offering the potential to preserve biological materials at ultra-low temperatures for extended periods. For example, the primary goal of cryopreservation is to maintain tissue viability, functionality, and structural integrity, enabling the tissues to be thawed and used effectively when needed. By preserving tissues at extremely low temperatures, cryopreservation halts biochemical reactions and cellular deterioration, essentially putting the tissues in a state of suspended animation. However, successful cryopreservation poses several challenges, including, for example, ice crystal formation during freezing, which can damage cells and disrupt tissue structures. Additionally, uniform distribution of cryoprotectant agents (also referred to herein as CPAs), which are substances used to protect tissues during freezing, should be achieved to preserve tissues effectively. Methods for restoring tissues that may be damaged also are lacking in the art due to the need for methods that can ensure sufficient distribution of therapeutics and/or regenerative materials into the tissue. As such, there is a need in the art for a method (and an apparatus/system for carrying out the method) that addresses these (and other) drawbacks to thereby unlock the full potential of cryopreserved tissues and/or restoration techniques for tissues, allowing advancements in medical treatments, transplantation procedures, and advancements in research and regenerative medicine.

### SUMMARY

Disclosed herein are embodiments of a loading apparatus, comprising: a loading mechanism for applying tension or compression to a biological material that becomes associated with the loading apparatus during use, the loading mechanism being manually or automatically operable; and an enclosure constructed with inert materials that contains the loading apparatus and that has a volume to accept a solution comprising a cryopreservation agent, therapeutic agent, restoration agent, or a combination thereof. In some embodiments, the loading apparatus further comprises one or more measurement features selected from a displacement sensor, a temperature sensor, a load sensor, or any combination thereof and wherein the one or more measurement features monitors parameters selected from temperature; concentration of the cryopreservation agent, therapeutic agent, restoration agent, or the combination thereof; tension; compression; and/or biological material dimensions during use of the loading apparatus.

Also disclosed herein are embodiments of a system, comprising: the loading apparatus according to the present disclosure; and a controller system in electrical communication with the loading apparatus, wherein the controller system regulates a loading cycle applied to the biological material. In some embodiments, the system further comprises: (i) one or more measurement features that monitors parameters selected from temperature, concentration, tension, compression, and/or biological material dimensions during use of the system; (ii) one or more imaging data modalities that provides magnetic resonance imaging (MRI) data, computed tomography (CT) data, positron emission tomography (PET) data, ultrasound data, X-ray data, optical scanning data, microwave imaging data, radar ultrasound data, photogrammetry data, elastography data, stereo-imaging data, or a combination thereof for evaluating distribution and attenuation of the cryopreservation agent, therapeutic agent, restoration agent, or the combination thereof within the biological material; (iii) one or more additional loading apparatuses and one or more additional enclosures, wherein each of the one or more additional loading apparatuses independently is contained within one of the one or more additional enclosures; (iv) a regulation component for regulating temperature, a CO₂ level, an O₂ level, and/or circulation of nutrients within the enclosure and the one or more additional enclosures; (v) one or more sensing devices for monitoring biological material properties selected from tensile changes, height changes, volume, and/or temperature, wherein the one or more sensing devices is physically associated with or coupled to the biological material, the loading apparatus (and/or the one or more additional loading apparatus), the enclosure (and/or the one or more additional enclosures), or a combination thereof; (vi) a plurality of tubes, ports, and liquid pumps that are independently physically or fluidly coupled to one or more of the enclosure (and/or the one or more additional enclosures) and that facilitate delivery of the cryopreservation agent, therapeutic agent, restoration agent, or the combination thereof into and/or out of the system; (vii) a data acquisition unit that is electronically coupled to the system; or (viii) any combination of two or more of (i)-(vii).

Also disclosed herein is a method, comprising: exposing a biological material to a cryopreservation agent, therapeutic agent, restoration agent, or a combination thereof; applying a load to the biological material using a loading apparatus; and removing the load from the biological material; wherein applying or removing the load facilitates distribution of the cryopreservation agent, therapeutic agent, restoration agent, or the combination thereof within the biological material. In some embodiments, the method further comprises obtaining a signal and quantifying an amount of the cryopreservation agent, therapeutic agent, restoration agent, or combination thereof that has permeated the biological material using one or more imaging data modalities selected from MRI data, CT data, PET data, ultrasound data, X-ray data, optical scanning data, microwave imaging data, radar ultrasound data, photogrammetry data, elastography data, or stereo-imaging data. In some additional embodiments of the method, the cryopreservation agent is used and the method further comprises: (i) cooling the biological material to a predetermined temperature to provide a cryopreserved biological material; (ii) storing the cryopreserved biological material in a cryogenic unit that maintains the cryopreserved biological material at a cryopreservation temperature; or (iii) a combination of (i) and (ii). In some such embodiments, the method further comprises: (i) thawing the cryopreserved biological material to provide a thawed biological material; (ii) exposing the thawed biological material to a restoration solution comprising a restoration agent; (iii) applying a load to the thawed biological material in the presence of the restoration solution using a loading apparatus; and (iv) removing the load from the thawed biological material; wherein applying or removing the load facilitates removing the cryopreservation agent from the thawed biological material and also facilitates uniform distribution of the restoration solution within the thawed biological material.

The foregoing and other objects, features, and advantages of the present disclosure will become more apparent from the following detailed description, which proceeds with reference to the accompanying figures.

### BRIEF DESCRIPTION OF THE DRAWINGS

FIG. 1 is a graph showing CPA concentrations at different positions within an IVD after 5 hours and 24 hours of undergoing a cryopreservation method.

FIG. 2 is an illustration showing an example of using diffusion to permeate a biological material with a cryopreservation agent, compared with permeating a biological material with a cryopreservation agent using compression and swelling.

FIG. 3 is a perspective view of a human spine, including a vertebral body 301, and an intervertebral disc tissue composite 400.

FIG. 4 is a perspective view of an intervertebral disc tissue composite, including the center of disc 402, endplates 401, and portions of the superior and inferior vertebral body 301.

FIG. 5 is a graph showing an example of IVD cryopreservation process stages.

FIG. 6 is a schematic diagram of an illustrative embodiment of a cryopreservation process.

FIG. 7 is a perspective view of an illustrative embodiment of a loading apparatus and the placement of an intervertebral disc tissue composite therein.

FIG. 8 illustrates one embodiment of a compression frame, enclosure, and apparatus within which an intervertebral disc is placed.

FIG. 9 is an illustration of a compression frame system as described herein.

FIG. 10 is a block diagram of an illustrative embodiment of a system according to the present disclosure.

FIG. 11 is a block diagram of a device configured to facilitate data processing, analysis, and control of a loading apparatus.

FIG. 12 shows mean signal intensity estimation of segmented scans of IVD using a Gaussian curve-fitting analysis.

FIG. 13 is a graph showing cell viability of nucleus pulposus (NP) cells in a monolayer culture exposed to CPA 1 at 4° C. for 24 hours (n=3).

FIG. 14 is a graph showing average DMSO penetration in IVDs (n=3) incubated in DMSO solution without prior compression of the IVDs.

FIG. 15 is a graph showing the effect of CPA type and incubation time on the viability of NP cells encapsulated in alginate.

FIG. 16 is a graph showing that DMSO penetration is much faster in IVDs (n=3) subjected to compression prior to incubation in the DMSO solution.

FIGS. 17A-17L are representative images of IVD sections stained using LIVE/DEAD™ stain after cryopreservation using various methods, compared to a fresh unfrozen control. The cryopreservation methods include: “no C.C.”—frozen without compression and without CPA; “CPA” frozen after loading CPA using diffusion alone; “C.C”—frozen after loading CPA using compression and subsequent swelling.

FIG. 18 is a graph showing mean cell viability in cryopreserved samples (n=4). The cryopreservation methods include: “no C.C.”—frozen without compression and without CPA; “CPA” frozen after loading CPA using diffusion alone; “C.C”—frozen after loading CPA using compression and subsequent swelling.

### DETAILED DESCRIPTION

### Overview of Terms

The following explanations of terms are provided to better describe the present disclosure and to guide those of ordinary skill in the art in the practice of the present disclosure. As used herein, “comprising” means “including” and the singular forms “a” or “an” or “the” include plural references unless the context clearly dictates otherwise. The term “or” refers to a single element of stated alternative elements or a combination of two or more elements, unless the context clearly indicates otherwise.

Unless explained otherwise, all technical and scientific terms used herein have the same meaning as commonly understood to one of ordinary skill in the art to which this disclosure belongs. Although methods and compounds similar or equivalent to those described herein can be used in the practice or testing of the present disclosure, suitable methods and compounds are described below. The compounds, methods, and examples are illustrative only and not intended to be limiting, unless otherwise indicated. Other features of the disclosure are apparent from the following detailed description and the claims.

Unless otherwise indicated, all numbers expressing quantities of components, molecular weights, percentages, temperatures, times, and so forth, as used in the specification or claims are to be understood as being modified by the term “about.” Accordingly, unless otherwise indicated, implicitly or explicitly, the numerical parameters set forth are approximations that can depend on the desired properties sought and/or limits of detection under standard test conditions/methods. When directly and explicitly distinguishing embodiments from discussed prior art, the embodiment numbers are not approximates unless the word “about” is recited. Furthermore, not all alternatives recited herein are equivalents.

Any theories of operation are to facilitate explanation, but the disclosed apparatuses, systems, materials, and methods are not limited to such theories of operation. Although the operations of some of the disclosed methods are described in a particular, sequential order for convenient presentation, it will be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed components and materials can be used in conjunction with other components and materials. Additionally, the description sometimes uses terms such as “produce” and “provide” to describe the disclosed methods. These terms are high-level abstractions of the actual operations that are performed. The actual operations that correspond to these terms will vary depending on the particular implementation and are readily discernible by one of ordinary skill in the art. Examples are described with reference to directions indicated as “above,” “below,” “upper,” “lower,” and the like. These terms are used for convenient description, but do not imply any particular spatial orientation.

To facilitate review of the various embodiments of the disclosure, the following explanations of specific terms and abbreviations are provided:

Biological Material: A material comprising a natural biological-based component or a synthetically-derived biological-based component. Biological materials can be engineered or extracted/harvested from a subject or sample. Exemplary, but non-limiting, biological materials include tissues, tissue composites, and/or tissue scaffolds.

Electronically Coupled: This term refers to a situation wherein two or more components are coupled so as to be in electronic communication with one another. Electronic communication can be facilitated with wires or it can be wireless.

Fluidly Coupled: This term refers to a situation wherein two or more components are coupled to one another directly or indirectly such that a fluid can flow from one component to the other component(s).

Load (or Loading): An amount of force, pressure, tension, or other action that is applied to a biological material. In some aspects of the disclosure, a load can comprise applying pressure to a biological material that is sufficient to compress the biological material to a shape and/or size that is different from that of its natural state, such as by reducing the size of the biological material. In some aspects of the disclosure, a load can comprise applying tension to a biological material by stretching it from its natural state.

Osmolarity: The concentration of solute particles in a solution and is typically measured in osmoles per liter (osmol/L) or milliosmoles per liter (mOsm/L).

Physically Associated (or Physically Coupled): This term refers to a situation where two or more components are associated so as to directly or indirectly physically touch.

### Introduction

Cryopreservation holds promise for preserving a wide range of biological material, including different tissue types, each with unique characteristics and considerations. These tissues can be broadly categorized into two main groups: vascular and avascular tissues. Vascular tissues, such as organs, muscles, and skin, possess a well-developed network of blood vessels that facilitate nutrient delivery and waste removal. The presence of blood vessels enables perfusion-based cryopreservation methods, where cryoprotectants (CPAs) are delivered through the circulatory system. On the other hand, avascular tissues, like cartilage, intervertebral discs (also referred to herein as an IVD or IVDs), and corneas, lack direct blood supply and rely on diffusion from nearby fluids for their metabolic needs. Cryopreserving avascular tissues presents distinct challenges due to limited nutrient supply and slower diffusion rates, and thus can benefit from the method described herein. By understanding the fundamental differences between vascular and avascular tissues, researchers and practitioners can tailor cryopreservation protocols to effectively preserve these diverse tissue types for various applications in medicine and research.

CPA delivery through the circulatory system is a widely used method in cryopreservation, especially for vascular tissues; however, avascular tissues, by definition, lack a direct blood supply and, as a consequence, are inherently recalcitrant or not very amenable to CPA delivery through the circulatory system. Without blood vessels to facilitate nutrient and CPA transport, avascular tissues have limited access to CPAs and, therefore, rely on alternative methods for preservation. On the other hand, even certain vascular tissues can also present challenges for CPA delivery via circulatory perfusion due to complex vascular structures, limited blood flow, or potential damage to delicate tissues during the process. As a result, both avascular and certain vascular tissues require specialized approaches, such as those discussed herein, to effectively introduce CPAs and ensure successful cryopreservation, preserving their viability and functionality for future use in medical applications and research.

Turning now specifically to illustrate cryopreservation of IVDs as a non-limiting example of biological material, it is well established that low back pain, often associated with IVD degeneration, is a prevalent condition that affects the quality of life of numerous individuals. This condition has been associated with disability, loss of income, loss of independence, depression, and loss of social support. It is estimated that every year over 700,000 Americans undergo invasive surgery, such as spinal fusion, to alleviate early and minor symptoms caused by IVD degeneration. Although spinal fusions have been rising, this procedure fails to treat degeneration. The procedure can also lead to further health complications, limiting the spine's range of motion and the patient's mobility. The failing spinal fusion outcomes can be attributed to the deficient guidelines outlining the best strategies to treat IVD degeneration. These strategies are left to the surgeon's discretion, considering personal preference, skill level, procedure complexity, and the patient's condition. Artificial IVDs made of various metals and polymers have emerged as an attractive alternative to treat IVD degeneration, maintaining the spine's range of motion and mobility. Moreover, these implants have been shown to limit degeneration at nearby IVDs compared to spinal fusion. However, one of the concerns associated with IVD implants is the deterioration of the material, limiting its lifetime, and requiring a replacement surgery. Replacement of an artificial IVD is also a significantly more complex procedure than the initial operation.

Implants of IVD allografts can thus provide an alternative to surgical strategies like spinal fusion, directly addressing degeneration and restoring spinal function. However, IVD allograft accessibility for research and clinical studies is limited due to challenges in long-term storage, compromising the viability of these specimens. One suggestion to overcome this challenge is to establish a bank of cryopreserved IVDs, which would enable IVD size matching between donors and recipients. A cryopreservation bank would also improve the logistics of clinical trials and IVD research by decreasing experiment costs and accommodating experimentation schedules. Cryopreservation provides a solution to the long-term storage of cells and tissues by lowering the specimen's temperature to a temperature sufficient to limit and preserve cellular functions (e.g., such as by maintaining the specimen at a temperature ranging from −80° C. to −196° C.). It also prevents damage to cells caused by the growth of intracellular ice crystals, the formation of extracellular ice, and changes in solute and osmotic levels. This process has revolutionized allograft clinical research by overcoming the increasing supply-demand imbalance for viable specimens.

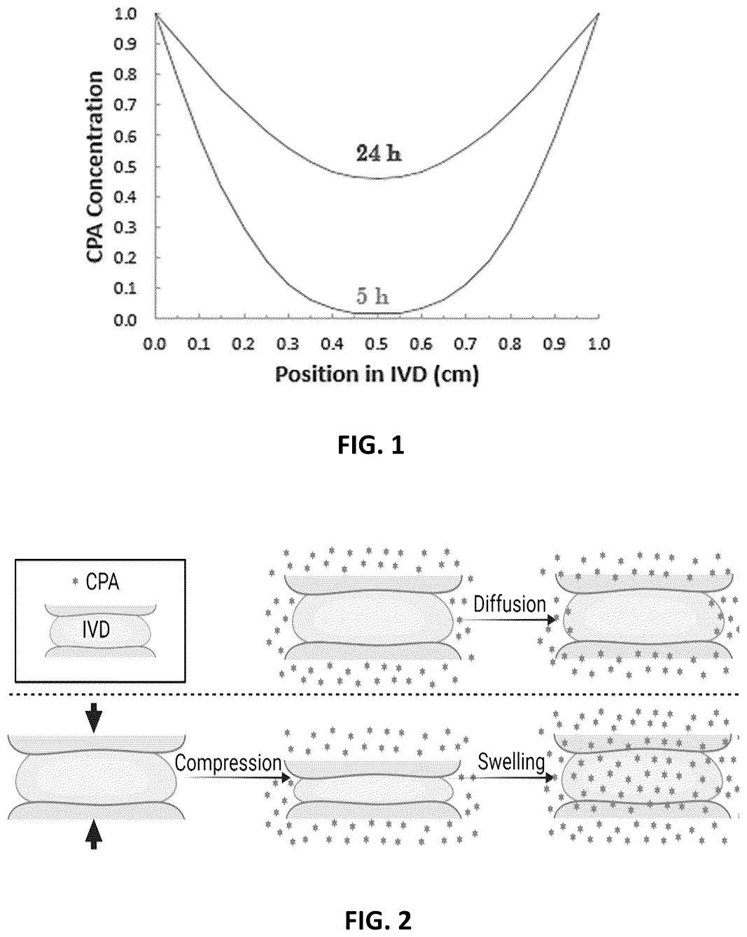

It is well recognized in the art that there are many problems facing the prospect of cryopreservation of sub-perfusable tissues (SPTs), including the non-limiting example of IVDs. Among these problems are the need for effective CPAs and their permeation throughout the tissue. Furthermore, most CPAs are cytotoxic, posing a risk to cell viability during the freezing process. Poor transport of the CPA during the cryopreservation procedure can result in incomplete penetration of the CPA within the tissue, leading to non-uniform distribution and potential tissue and cell damage. As can be seen by FIG. 1, it can take significant time for a CPA to permeate into depths of a tissue sample without the assistance of the technology described herein. FIG. 1 shows that it takes 24 hours for increased concentrations to permeate the depth of a tissue sample as compared to the minimal permeation that takes place after 5 hours. Consequently, prolonged CPA exposure of cells near the surface of the tissue, as shown by the extended timeframe, poses an increased risk of cell damage, while cells in the center are unprotected from freezing damage. This damage can further compromise the structural integrity of the tissue. Therefore, current cryopreservation techniques are highly dependent on CPA permeation rates and cooling. Accordingly, additional systems, devices, and methods for cryopreservation of tissue that minimize tissue and cellular damage are desirable.

With reference to the IVD as an exemplary biological material, it can be noted that the IVD, as the largest avascular structure in the human body, presents challenges for efficient delivery and expulsion of a CPA, therapeutic agent, restoration agents, or a combination thereof. The transport of such agents within IVD is constrained by the large size of the composite tissue and the avascular nature, which limits the mechanisms available for agent movement. Additionally, given the significant diffusion distance within the IVD, achieving uniform permeation throughout the tissue becomes challenging. The combination of the IVD's size and avascular nature restricts agent transport, thereby impeding uniform agent penetration and distribution during conventional cryopreservation methods. Additionally, the charged nature of the ECM within the IVD and the potential calcification of the bony endplates further complicate the distribution of CPAs. The ECM of IVDs contains charged molecules, such as proteoglycans, which can create electrostatic barriers that hinder the movement of charged CPAs. Therefore, conventional cryopreservation methods struggle to achieve full and even permeation and distribution of, for example, CPAs within SPTs, including the non-limiting example of IVDs. Such methods primarily rely on diffusion for CPA transport, and diffusion is a passive process that is dependent on random molecular movement.

Additionally, diffusion can be slow and inefficient, especially in large tissues like the IVD, making it challenging for CPAs to effectively permeate and reach all regions of the tissue uniformly. The limited diffusion capacity hampers the comprehensive distribution of CPAs, potentially leading to inadequate preservation outcomes, tissue damage, and cell death.

Other cryopreservation methods known in the prior art, such as vitrification, offer the advantage of preventing ice crystal formation and preserving tissue integrity; however, these methods present challenges in achieving full permeation and uniform distribution of CPA. High CPA concentrations required for vitrification may pose risks of cellular toxicity and osmotic stress, potentially compromising tissue viability. Non-uniform CPA distribution can adversely affect preservation outcomes and compromise tissue viability.

The above-mentioned problems limit the effectiveness of cryopreservation using conventional methods, especially those that rely on passive diffusion of CPAs. Moreover, the effective application of conventional cryopreservation methods may be limited to small tissue composites, tissues of lower complexity, and tissues with inherently generous transport properties.

In addition to cryopreservation, the ability to efficiently and effectively revive and/or restore biological materials exists as a need in the art. For example, tissues that are damaged could benefit from a method whereby the tissue can be treated so as to be restored back to a normal healthy state. Some tissues are not easily treated while in the body but could be treated efficiently by extracting the tissue and exposing it to a method or operable system/device capable of revising or restoring the tissue back to a healthy state where it can be implanted back into the location from which it was extracted or transplanted into a recipient.

Disclosed herein are embodiments of a method for cryopreserving and/or restoring a biological material. In particular aspects of the disclosure, a loading apparatus is described that comprises a loading mechanism for applying a load (e.g., tension or compression) to a biological material that becomes associated with the loading apparatus during use, wherein applying or removing the load facilitates the ability to fully disperse and remove a CPA, therapeutic agent, and/or restoration agent within the biological material. Additional components of the loading apparatus are described herein. System embodiments comprising the loading apparatus also are described. The method embodiments disclosed herein utilize the loading apparatus and/or system embodiments disclosed herein to facilitate cryopreserving and/or restoring biological materials more efficiently than conventional methods. The method disclosed herein is able to achieve a desirable dispersion of a CPA, therapeutic agent, and/or restoration agent within the biological material with minimal cell death and/or within timing parameters needed for effective treatment. As such, the method is able to achieve successful preservation and/or restoration ensuring biological material viability and functionality for applications in medicine and research.

### Method of Cryopreservation and/or Restoration

Aspects of the present disclosure concern a method for cryopreservation and/or restoration of biological materials. In particular disclosed aspects, the method comprises exposing a biological material to a cryopreservation agent, therapeutic agent, restoration agent, or a combination thereof; applying a load to the biological material using a loading apparatus; and removing the load from the biological material, wherein applying or removing the load facilitates uniform distribution of the cryopreservation agent, therapeutic agent, restoration agent, or the combination thereof within the biological material. The load can comprise any sufficient force, pressure, tension, or other action that causes the biological material to deform, with representative loads being described herein.

In some aspects of the present disclosure, the biological material can be exposed to the CPA, therapeutic agent, restoration agent, or the combination thereof by applying the agent either directly or proximally to the biological material. In some aspects of the disclosure, the biological material can be exposed to a bath comprising the CPA, therapeutic agent, restoration agent, or the combination thereof.

Applying the load to the biological material, such as in the case of compressing the biological material, facilitates penetration and distribution of the agent into the biological material. In some aspects, the load comprises compressing the biological material to a smaller size or shape different from its natural state. In other aspects, the load comprises stretching the biological material.

Removing the load can comprise any action sufficient to remove the force or action that caused the biological material to deform. In some aspects of the disclosure, removing the load can comprise allowing the biological material to decompress back to its natural state. In yet other aspects of the disclosure, removing the load can comprise releasing the biological material from a stretched state back to its natural state.

Decompressing or stretching the biological material facilitates “free-swelling” of the biological material and creates a “suction” effect. An illustrative schematic of the free-swelling phenomenon disclosed herein is shown in FIG. 2, which shows the process in the context of using compression and decompression as the load applied to the biological sample. This effect augments the active transport of agents into the biological material, harnessed through the biological material's inherent ability to recover its original dimensions. Without being limited to a single operating theory, it currently is believed that this mechanism establishes a favorable pressure gradient, which aids in the directed movement of agents into the biological material, especially for tissues, such as cartilage, where agent transport can be challenging. In some aspects of the disclosure, free-swelling mediated transport can be used to improve permeation of CPAs, therapeutic agents and/or restoration agents into biological materials. In some aspects of the disclosure, this free-swelling technique can be used to allow greater than 0% to 100% of the CPA (e.g., 50% to 100%, or 75% to 100%), therapeutic agent, and/or restoration agent to permeate the biological material within a time frame of 30 minutes to 24 hours, such as 2 to 8 hours, or 2 to 4 hours.

In particular aspects of the present disclosure, the method can comprise one or more cycles wherein load is applied and removed. In some aspects, the steps of applying the load and removing the load may be repeated for multiple cycles, such as from two cycles to 200 cycles, two cycles to 100 cycles, two cycles to 50 cycles, two cycles to 20, or two cycles to 10 cycles. The amount of load that can be applied in the method can be selected based on the type of biological material being used. In some aspects of the disclosure, the load can be a static load or a dynamic load. In embodiments using static loading, the amount of pressure or tension applied can range from 0.01 MPa to 8 MPa, such as 0.05 MPa to 5 MPa, or 0.05 MPa to 0.2 MPa, or 0.05 MPa to 0.1 MPa. In embodiments using dynamic loading, the amount of pressure or tension applied can range from 0.01 MPa to 8 MPa, such as 0.05 MPa to 0.2 MPa, or 0.05 MPa to 0.1 MPa and the frequency can range from greater than 0 Hz to 1 Hz, such as 0.001 Hz to 0.5 Hz, or 0.1 Hz to 0.5 Hz, or 0.1 Hz to 0.15 Hz. Load can be applied for a time period ranging from 30 minutes to 24 hours, such as 2 to 8 hours, or 2 to 4 hours.

In particular aspects of the disclosure, the disclosed method facilitates an increase in the amount of the cryopreservation agent that is distributed throughout the biological material within a time period where minimal to no cell death occurs in the biological material. As CPAs are known to be toxic upon reaching certain parameters, ensuring that the CPA is sufficiently distributed within the biological material within a suitable time frame is effective in reducing cell toxicity/death. In some aspects of the disclosure directed to using cryopreservation agents, the method facilitates distributing the CPA into the biological material within a time frame that avoids cell death, such as within 6 to 48 hours, or 6 to 12 hours, or 6 to 8 hours after exposing the biological material to the CPA. In particular embodiments, over 90% of the CPA was sufficiently distributed within the biological material within three hours after exposing the biological material to the CPA utilizing a method wherein compression was used to facilitate free swelling-promoted distribution of the CPA.

In some aspects of the disclosure, the method can further comprise obtaining a signal (for real-time and/or delayed monitoring) and quantifying an amount of the cryopreservation agent, therapeutic agent, restoration agent, or combination thereof that has permeated the biological material using one or more imaging data modalities selected from MRI data, CT data, PET data, ultrasound data, X-ray data, optical scanning data, microwave imaging data, radar ultrasound data, photogrammetry data, elastography data, stereo-imaging data, or combinations thereof. Additionally, other methods may be used to measure the concentration of agents inside the biological materials. One such technique involves obtaining tissue samples from the biological material. Such samples can be obtained using biopsy. Then, the samples may be homogenized to break down the structure. Subsequently, the agent of interest is extracted from the biological material homogenate using suitable solvents or extraction methods. The concentration of the agent can then be quantified using analytical techniques, such as high-performance liquid chromatography (HPLC), liquid chromatography-mass spectrometry (LC-MS), or gas chromatography-mass spectrometry (GC-MS), and chemical assays, such as enzyme-linked immunosorbent assay (ELISA). Additionally, microdialysis can be employed to measure the concentration of the agent inside the biological material. This method entails the insertion of a microdialysis catheter into the biological material to collect extracellular fluid. The agent of interest diffuses from the biological material into the probe's dialysate, which is then collected and subjected to analysis for determining agent concentration. Also, an implantable sensor might be used to measure changes in agent concentration. Moreover, the concentration of the agent in solution, such as the cryopreservation agent, therapeutic agent, restoration agent, or their combinations, can be measured using any of the methods mentioned earlier, as well as other appropriate analytical techniques. By quantifying the agent concentration in the solution, researchers can then employ calculations and mathematical models to estimate or deduce the level of agent present within the biological material.

In some aspects of the present disclosure, the method can further comprise preparing the biological material by isolating the biological material from surrounding tissue, bone, or skin. In some particular aspects of the disclosure, the biological material may be extracted from any surrounding tissue, bone, or skin using techniques described herein.

In yet additional aspects of the disclosure, particularly those utilizing a CPA, the method can further comprise cooling the biological material to a predetermined temperature to provide a cryopreserved biological material and storing the cryopreserved biological material in a cryogenic unit that maintains the cryopreserved biological material at a cryopreservation temperature. In some aspects of the disclosure, cooling the biological material can be achieved using a temperature ramp protocol wherein the temperature surrounding the biological material is lowered at a cooling rate of −0.5° C. per minute to −10° C. per minute, or −1° C. per minute to −5° C. per minute, or −1° C. per minute to −2° C. per minute until a temperature of −20° C. to −80° C. is reached. At this point, the biological material is transferred to an environment held at a temperature ranging from −80° C. to −196° C., such as to an environment comprising liquid nitrogen or a −80° C. freezer. In other aspects of the disclosure, the temperature can be reduced rapidly to a temperature below −100° C. In such aspects, the biological material can be vitrified without forming ice crystals. In some aspects of the disclosure, the biological material can be exposed to the CPA prior to cooling by exposing the biological material to a CPA bath that comprises the CPA and saline.

In some aspects of the disclosure, the biological material can be compressed just prior to cooling to the cryopreservation temperature for storage. This results in storage of the material in a compressed state. Upon thawing, the biological material can be placed in a CPA-free (or low-CPA) solution that may contain therapeutic or restorative agents, where it will free-swell, drawing this external solution into the material.

In some such aspects using a CPA for cryopreservation, the method can further comprise a thawing process. The thawing process can comprise thawing the cryopreserved biological material to provide a thawed biological material; exposing the thawed biological material to a restoration solution comprising a restoration agent; applying a load to the thawed biological material in the presence of the restoration solution using a loading apparatus; and removing the load from the thawed biological material, wherein applying or removing the load facilitates expelling the CPA from the biological material and further facilitates uniform distribution of the restoration solution within the thawed biological material. In some aspects of the disclosure, the system and/or loading apparatus, along with method steps disclosed herein for use during the cryopreservation phase of a method may be adapted and utilized in the thawing process. For example, temperature-controlled environments, cryogenic storage units, and specialized containers designed for cryopreservation can be employed to control the thawing process efficiently. In such aspects of the disclosure, at least certain steps of the thawing process can be carried out in the presence of a restorative solution comprising saline and any desired restoration agent. By compressing (or stopping stretching of) the biological material, the CPA can be expelled from the biological material. The biological material is then allowed to decompress (or relax) back to its natural physical state so as to facilitate absorption and dispersion of any restoration agents. These techniques assist in the rapid diffusion of CPAs out of the biological material and further facilitate distributing the restoration agents throughout the biological material, thereby expediting the restoration process and reducing the risk of cellular damage associated with prolonged exposure to CPAs. Loading parameters and components used for a thawing process can be the same as or similar to those used to introduce the CPA, as described herein.

In particular aspects of the disclosure, the restorative solution used in the described method can be selected to have a particular osmolarity match with the biological material, such that the restorative solution has the same (or substantially the same, such as within 1-10%) osmolarity as the target osmolarity of the biological material when reintroduced to the desired environment (for example, after transplantation into a subject). The restorative solution also is isotonic and thus has the same (or substantially the same, such as within 1-10%) osmotic pressure as cells of the biological material, which facilitates preventing water from either entering or leaving the cells, thus avoiding osmotic stress and maintaining cell integrity. The restorative solution also is sterile so as to avoid any contaminants or pathogens into the biological material and exhibits biocompatibility such that it does not elicit any significant immune response or adverse reactions when in contact with the biological material or any subject in which the biological material is implanted. The restorative solution also is PH-balanced within a physiological range to ensure compatibility with the biological material and prevent any pH-induced damage to cells or tissues. In some aspects of the disclosure, the restorative solution further comprises a therapeutic agent. In such aspects, the therapeutic agent typically is selected or modified so as to be soluble in the restorative solution and capable of permeating the biological material. Restorative solutions described herein can be used in method embodiments that do not utilize cryopreservation, such as restoration method embodiments described herein.

To achieve controlled thawing, the cryopreserved biological material can be placed in a temperature-controlled environment or system. The temperature control ensures that the thawing process occurs in a consistent and repeatable way, minimizing the risk of cellular damage or structural alteration. In some aspects of the disclosure, the material can be thawed by placing it in a water bath (or other temperature-controlled environment) that is maintained at a temperature between −20° C. to 40° C., such as 20° C. to 40° C., or 37° C. to 40° C. In some aspects of the disclosure, thawing the cryopreserved biological material comprises using a temperature ramp, wherein the temperature is raised at a ramp rate ranging from 0.1° C./min to 100° C./min. The ramp rate can be varied to achieve different rates of warming in different temperature intervals.

In some aspects of the disclosure, the biological material is pre-loaded with magnetically responsive nanoparticles, such as iron oxide nanoparticles, to enable rapid and uniform heating by applying an alternating magnetic field. The nanoparticles can be loaded into the biological material by applying and removing a load, using methods such as those described above.

As described above for aspects of the disclosure utilizing a CPA, the biological material can be stored at predetermined temperatures in specialized cryogenic storage units or facilities; however, there are situations where a cryopreserved biological material should be restored to its original functional state for various purposes. In some aspects of the disclosure, and as described above, the cryopreserved biological material can be thawed so as to return it to its normal physiological temperature and ensure the viability and integrity of the biological material during the method.

### Biological Materials and Agents

In some aspects of the disclosure, the biological material according to the present disclosure comprises tissues (including, for example, sub-perfusable tissues also referred to herein as “SPTs”), tissue composites, tissue scaffolds, or any combination thereof. In some aspects of the disclosure, the biological material is an SPT selected from an avascular tissue, vascular tissue, compound anatomical structure that may contain both vascular and avascular tissues, and any other tissue described herein that are not readily amenable to circulatory CPA transport.

In some aspects of the disclosure, avascular tissues can include, but are not limited to, cartilage, cornea, meniscus, tendons, ligaments, IVDs, hyaline cartilage, elastic cartilage, fibrocartilage, epiphyseal cartilage, articular cartilage, tendon sheaths, cartilaginous endplates, avascular necrosis (AVN) lesions, avascular zones in organs, ocular lens, and combinations thereof.

In some aspects of the disclosure, vascular tissues can include, but are not limited to, heart, liver, kidney, muscle (skeletal, smooth, and cardiac), lung, skin, brain, spleen, pancreas, intestinal, ovarian, testicular, adipose, bone marrow, uterine, retinal, adrenal, thyroid, thymus, or combinations thereof.

In some aspects of the disclosure, tissue composites and/or tissue scaffolds are composite materials that comprise a structural component that is or can be associated with a biological material. In some aspects of the disclosure, a tissue composite or scaffold can comprise a structural component created with advanced manufacturing techniques such as additive manufacturing (3D printing), nanomanufacturing, microfabrication, precision machining, laser cutting, electrochemical machining (ECM), advanced robotics, advanced casting techniques, smart materials and adaptive manufacturing, bioprinting, and hybrid manufacturing. Exemplary structural components of a tissue composite and/or scaffold can include, but is not limited to synthetic polymers, natural polymers, biocompatible ceramics, decellularized tissues, self-assembling peptides, hydrogels, composite scaffolds, nanofibrous scaffolds, 3D printed scaffolds, electro-spun scaffolds, nanocomposite scaffolds, porous scaffolds, thermo-responsive scaffolds, chitosan-based scaffolds, alginate-based scaffolds, silk-based scaffolds, gelatin-based scaffolds, polymeric microspheres, cellulose-based scaffolds, nanocellulose scaffolds, fibrin-based scaffolds, calcium phosphate cement, titanium-based scaffolds, polymeric beads, nanoparticle-embedded scaffolds, polymeric foam scaffolds, collagen-glycosaminoglycan scaffolds, biodegradable elastomers, photo-crosslinked hydrogels, and composite microspheres. The tissue component of a tissue composite and/or scaffold can further comprise organoids, spheroids, co-cultures, bioprinted constructs, decellularized extracellular matrix, and combinations thereof.

SPTs according to the present disclosure typically refer to those tissues and biological structures that lack a direct blood supply, have complex vascular structures, restricted blood flow, limitations in blood supply, or exhibit other factors that hinder effective CPA delivery through the circulatory system. Such SPTs present challenges, using conventional cryopreservation techniques, in achieving optimal CPA distribution through their vascular network (if any vascularization is present at all) and thus such SPTs benefit from the method, apparatus, and system embodiments described herein. SBTs may have complex vascular structures, restricted blood flow, or other factors that hinder the effective and non-toxic transport of CPAs to all areas of the tissue.

In exemplary aspects of the disclosure, SPTs used in method embodiments that have limited (or a minimal level of) vascularization can include those listed below, with such SPTs exhibiting incompatibility with conventional cryopreservation and/or restoration methods for reasons noted below:
Full Thickness Skin Grafts: Skin grafts are vascular tissues used for wound healing and reconstructive surgeries. Conventional cryopreservation and/or restoration methods do not effectively perfuse the graft with CPAs or restoration agents due to the complex network of blood vessels and lack of access to a cannulation site that would effectively perfuse the tissue;
Muscle Grafts: Muscle tissue is highly vascularized, but the complex network of blood vessels can make perfusion challenging using conventional methods;
Ovarian Tissue: Oocytes and the surrounding ovarian tissue are crucial for fertility preservation. Conventional approaches to cryopreservation are limited in their ability to perfuse CPAs into ovarian tissues.
Articular Cartilage: While articular cartilage is avascular, it is part of a larger vascularized joint structure. In some cases, direct (vascular) perfusion might be challenging or impossible. For example, conventional methods are limited in preserving articular cartilage, despite a documented need for such preservation in research, regenerative medicine, and other applications.
Tendon and Ligament Grafts: Tendons and ligaments are connective tissues that play a crucial role in joint stability. Cryopreservation of these tissues using the method, apparatus, and/or system embodiments disclosed herein can facilitate tissue engineering and transplantation procedures.

Also by way of example but not limitation, some examples of SPTs include strictly avascular tissue. Avascular tissues are tissues that lack a direct blood supply and, as a result, are generally more challenging to preserve compared to vascular tissues. The absence of blood vessels in avascular tissues means that they rely on diffusion from nearby blood vessels or surrounding fluids for their metabolic needs and waste removal. This unique characteristic presents several distinct features and challenges, particularly when it comes to tissue cryopreservation and/or restoration using conventional methods. For example, avascular tissues have a limited ability to access nutrients from the circulatory system due to the absence of blood vessels, which makes it difficult to deliver CPAs, therapeutic agents, and/or restoration agents uniformly throughout the tissue during the preservation and/or restoration process. In cryopreservation, CPAs are needed to protect tissues from ice crystal formation during cooling; however, avascular tissues have slow diffusion rates, making it difficult for CPAs to penetrate deeply into the tissue and achieve uniform distribution. Slow diffusion leads to potential uneven distribution of CPAs and may result in regions of the tissue being inadequately protected during cooling. Similar effects (slow diffusion and/or non-uniform distribution) can be seen when attempting to restore or treat a tissue using a therapeutic agent and/or a restoration agent. In some aspects, avascular tissues are especially vulnerable to damage from ice formation. As such, without proper distribution of CPAs, therapeutic agents, and/or restoration agents, avascular tissues are at a higher risk of damage (e.g., from ice crystal formation during cooling) and/or inadequate treatment leading to undesired cell death and/or inability to use the restored tissue. Also, in the case of cryopreservation, ice crystals can damage cellular structures and disrupt tissue integrity, leading to reduced viability upon thawing and/or the slow diffusion of CPAs and the potential for ice formation can cause cellular damage and osmotic stress in avascular tissues, which consequently affects their overall viability and functionality after cryopreservation.

As noted elsewhere in this document, there are many categories of avascular tissue (and compound structures containing avascular tissue, and SPTs in general) that may become of interest for cryopreservation. Common examples of primarily avascular SPTs include, without limitation:
Intervertebral Discs (IVDs): Cartilaginous structures found between the vertebrae in the spine, acting as shock absorbers and allowing flexibility of the spine. They are avascular, relying on diffusion from surrounding tissues for nutrition and waste removal. IVDs have a complex structure composed of three main components: the annulus fibrosus, the nucleus pulposus, and the cartilaginous endplates.
Articular Cartilage: Found in joints, articular cartilage allows smooth movement of bones. Its avascular nature makes it susceptible to damage during cryopreservation, affecting joint health.
Cornea: The avascular outer layer of the eye, the cornea, is responsible for focusing light onto the retina. Cryopreservation of corneas is used for transplantation and restoring vision.
Meniscus: These crescent-shaped structures in the knee joint help with load distribution and shock absorption. Cryopreservation can be used to preserve meniscus function after injuries.
Tendons and Ligaments: These avascular connective tissues support and stabilize joints. Cryopreservation can be valuable for tissue engineering and regenerative medicine applications.

All these avascular tissues, as with SPTs generally, present unique challenges for cryopreservation due to, for example, limited nutrient supply, slow diffusion, vulnerability to ice formation, and potential cellular damage. IVDs serve as an illustrative, non-limiting example of these challenges in SPTs. Similar concerns apply to other important SPT tissues found in various parts of the body. Overcoming these challenges can facilitate cryopreservation and the potential use of avascular tissues in regenerative medicine, transplantation, and research applications. In particular aspects of the disclosure, the method is particularly useful for SPTs that are not amenable to traditional circulatory CPA delivery, such as avascular tissues or certain vascular tissues with complex structures that hinder efficient perfusion. The method facilitates achieving uniform CPA distribution, ensuring successful preservation of the tissue's viability and functionality upon thawing. Further, a host of SPTs, including biological structures and tissue types, have similar concerns as those discussed herein with respect to IVDs and conventional methods known in the art for cryopreserving such materials, and it is intended that all of these SPTs, biological structures and tissue types are included within the scope of present disclosure as being applicable in the disclosed method, apparatus, and system embodiments.

In embodiments using a CPA, the CPA can be selected from dimethylsulfoxide (DMSO), ethylene glycol, propylene glycol, butanediol, cyclohexanediol, diethylene glycol, 3-methoxy-1,2 propanediol, glycerol, formamide, acetamide, propionamide, lactamide, malonamide, dimethylformamide, methylacetamide, dimethylacetamide, betaine, monoacetin, triacetin, ice recrystallization inhibitors, antioxidants, apoptosis inhibitors, metformin, alginate, egg yolk, polyethylene glycol (PEG), polyvinylpyrrolidone (PVP), raffinose, sucrose, trehalose, ethanol, ficoll, polyvinyl alcohol (PVA), methylsulfonylmethane (MSM), polyethyleneimine (PEI), hydroxypropyl cellulose, dextran, or combinations thereof. In some aspects of the disclosure, the CPA can be used in combination with a visualization agent (e.g., a contrast or stain agent). In particular aspects of the disclosure, the visualization agent can be selected from iodinated contrast agents, such as iohexol, iopamidol, ioversol, iodixanol, iomeprol, iopromide, iopentol, iotrolan, gadobutrol, gadoterate meglumine, gadoteridol, gadodiamide, gadopentetate dimeglumine, gadoteridol, barium sulfate, perfluoropropane lipid microspheres, gold nanoparticles, silica nanoparticles, silver nanoparticles, sulfur hexafluoride microbubbles, ferumoxides, ferucarbotran, ferumoxytol, fluorescein diacetate, calcein AM, STYO green, propidium iodide, ethidium bromide, methylene blue stain, green stain, brilliant cresyl blue stain, or combinations thereof.

In some aspects of the disclosure, the therapeutic agent may be selected from anti-inflammatory agents, immunosuppressive agents, immunomodulatory agents, vaccines, chondrogenic differentiation agents, cancer-specific inhibitors, gene therapies, anti-aging, anti-fibrotic agents, cell-survival signaling agents, pain management agents, osteogenesis agents, pan-caspase inhibitors, senolytics/senotherapeutics, antiapoptotic agents, or antibiotic drugs. In certain examples, the therapeutic agent can be selected from gboxin, prednisone, cyclosporine, tacrolimus, azathioprine, mycophenolate mofetil, sirolimus, everolimus, alemtuzumab, antithymocyte globulin, thymoglobulin, ATGAM (equine), antibodies, glucocorticoids, immunoglobulin G, rituximab, belatacept, iRNA, mRNA, siRNA, lipid nanoparticles, p53 siRNA-cy3, enkephalins, RG-7112, o-vanillin, dasatinib, quercetin, fisetin, luteolin, curcumin, curcumin analog EF24, navitoclax (ABT263), A1331852, A1155463, geldanamycin, tanespimycin, alvespimycin, and other HSP90 inhibitors, piperlongumine, FOXO4-related peptide, nutlin3a, cardiac glycosides such as ouabain, proscillaridin A, and digoxin, aspirin, transforming growth factor-beta, HGF (Hepatocyte Growth Factor), indoleamine 2,3-dioxygenase (IDO), PGE2 (prostaglandin E2), nitric oxide (NO), IL-6 (interleukin-6), IL-10 (interleukin-10), TNF-α (Tumor Necrosis Factor-alpha), IFN-γ (Interferon-gamma), FOXP3 (Forkhead box protein P3), CTLA4 (Cytotoxic T-lymphocyte-associated protein 4), GITR (Glucocorticoid-induced tumor necrosis factor receptor), TGF-β1 or TGF-β3 (Transforming Growth Factor-beta 1 or 3), IGF-1 (Insulin-like Growth Factor 1), FGF-2 (Fibroblast Growth Factor 2), BMP-2, BMP-4, BMP-6, BMP-7 (Bone Morphogenetic Proteins 2, 4, 6, 7), ascorbic acid, β-glycerophosphate, vitamin D3, VEGF (Vascular Endothelial Growth Factor), IGF-1 (Insulin-like Growth Factor 1), N-acetyl-cystein, acetaminophen (paracetamol), nonsteroidal anti-inflammatory drugs (NSAIDs), SNRIs, tricyclic antidepressants (TCAs), benzyloxycarbonyl-Val-Ala-dl-Asp-fluoromethylketone (ZVAD).

In some aspects of the disclosure, the restoration agent can be a nutrition agent, such as amino acids, vitamins, minerals, sugars (e.g., glucose), salts (e.g., sodium chloride), pH buffers (e.g., HEPES), Serum (e.g., calf serum), growth factors, hormones, antibiotics/antimycotics, lipids, gases (e.g., carbon dioxide), chelating agents, buffering agents, water, or combinations thereof.

The CPA, therapeutic agent, and/or restoration agent can be used at concentrations sufficient to facilitate the intended purpose. For example, CPAs can be used at concentrations that facilitate cryopreservation of a biological material. In particular aspects of the disclosure, the CPA can be provided at a concentration ranging from 5% to 60%, such as 5% to 50%, or 10% to 25%, or 10% to 15%. Therapeutic agents can be used at a concentration that facilitates reducing swelling, inflammation, pain, transplant rejection, and/or medical treatment of an underlying condition. Such amounts can be determined using knowledge in the art with the benefit of the present disclosure. In some aspects of the disclosure, the therapeutic agent can be an antibiotic and can be used at a concentration ranging from greater than 0% to 10%, such as 1% to 5%, or 1% to 3%. In aspects of the disclosure utilizing a restoration agent in the method, the restoration agent can be used at a concentration sufficient to revive a biological material and/or to maintain it in a healthy state. In some particular aspects, the restoration agent is used at a concentration that provides nutrition to the biological material, such as concentrations ranging from 5% to 90%, such as 65% to 90%, or 70% to 80%.

### Cryopreservation Method Embodiments

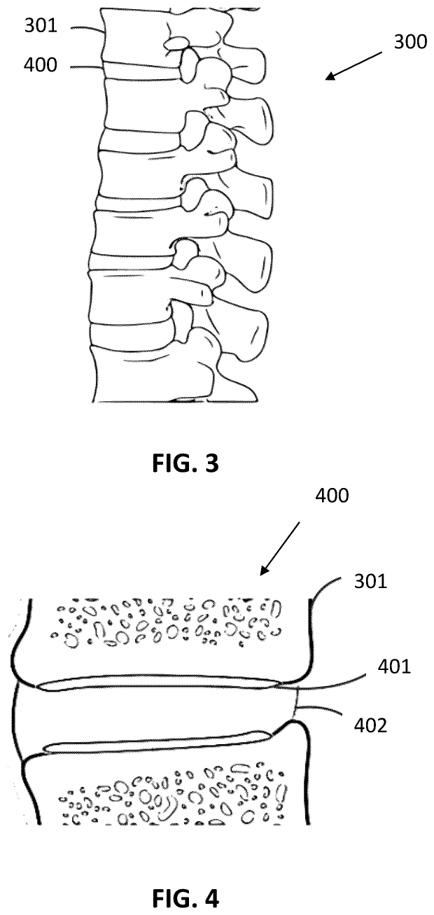

Another representative example of a method is described herein. In this example, a biological material isolation step according to the present disclosure is utilized as follows with reference to IVD tissue composite 400, as shown in FIGS. 3 and 4. The cryopreservation process begins with the receipt of an IVD tissue composite 400 from a tissue source, such as a body or procurement facility. The IVD is carefully prepared by separating the spinal column 300 from the source. This process may also include trimming and dissection, ensuring the removal of any unwanted adjacent tissues or structures (e.g., inferior vertebral body 301), and isolating the target tissue. In some aspects of the disclosure, it is useful to keep some bone (e.g., inferior vertebral body 301) attached to the endplates 401 and the body of the IVD 402 to keep it intact. To maintain a sterile environment and prevent contamination, the dissected IVD is wrapped in sterile gauze soaked in betadine or other disinfecting agents. Following disinfection, the IVD is washed three times in PBS. Prior to the cryopreservation step, the IVD tissue can be incubated in cell culture media at 37° C. for at least 24 hours. This incubation step can be utilized to provide a controlled environment conducive to cell recovery and tissue stabilization post the dissection process.

Next, IVD tissue properties, such as mass and height, are measured. These measurements serve as baseline data to calculate the needed load for the compression cycles, evaluate the effectiveness of the cryopreservation process, and provide insights into the tissue's integrity and stability during subsequent analyses.

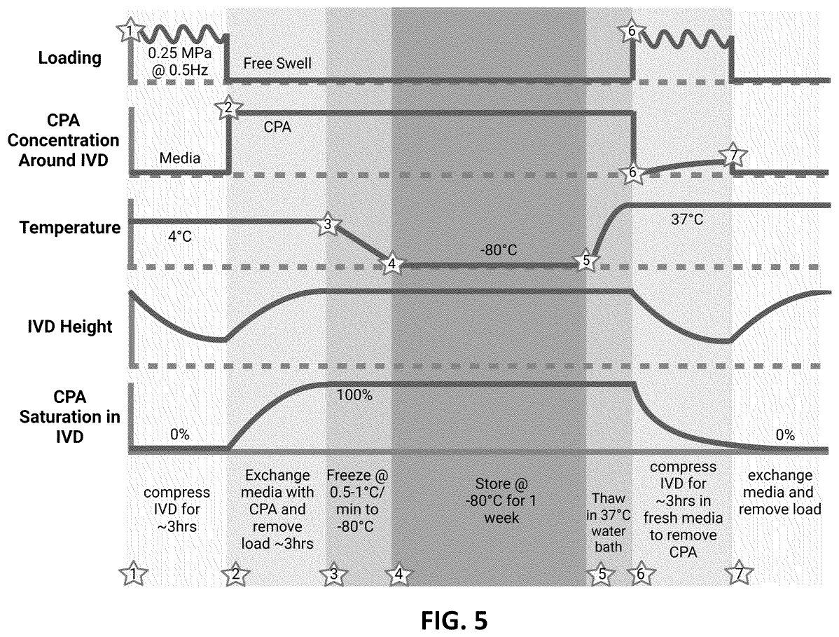

Once the IVD 400 is isolated and processed for cryopreservation, it is placed in a loading apparatus 700. Next, the IVD 400 is subjected to controlled compression using the loading apparatus. The following steps are performed at 4° C. to prevent cytotoxic effects of the CPA solution. The appropriate load is calculated by determining the force needed to produce 0.1 MPa utilizing the IVD dimensions measured previously. The appropriate load is applied at 1 Hz frequency for three hours. Next, a formulated cryoprotectant (CPA) solution is prepared and introduced into the loading apparatus. In some examples, this CPA solution contains a mixture of dimethyl sulfoxide (DMSO); propylene glycol, ethylene glycol, or a combination thereof; and a cell culture medium (e.g., DMEM). The precise proportions of these components are tailored to promote tissue permeation and preservation. In this particular example, the following ratios were used: 69% DMEM, 10% FBS, 10% DMSO, 10% propylene glycol, and 1% antibiotic/antimycotic. Other contentions and ratios may also be used. Next, a decompression phase is applied for three hours. This phase allowed the tissue to recover to its original shape and size. An overview of this process (and other certain steps described herein) is shown schematically in FIG. 5. If necessary, multiple loading and decompression cycles can be used, in which the CPA concentration is gradually increased with each cycle.

In between each loading cycle, the CPA concentration is measured to monitor the permeation progress. This can be done using osmolarity measurements to determine the CPA levels in the solution, and a material balance to estimate the corresponding CPA concentration in the IVD. CT imaging may also be used to evaluate whether full permeation of the IVD has been fulfilled.

Once CPA permeation is achieved, the target tissue undergoes controlled cooling to sub-zero temperatures at a freezing rate of −1° C. per minute. This gradual cooling prevents forming damaging intracellular ice crystals and helps preserve the tissue's structural integrity and functionality during cryopreservation. The tissue is then moved to long-term storage, which usually includes storage temperatures lower than −80° C.

To restore the cryopreserved IVD, it is thawed by placing it in a bath. Next, the IVD is returned to the loading apparatus to remove the CPA from the tissue. A similar procedure to the one described earlier is performed, applying cyclical compression and decompression at 4° C. To ensure the complete removal of the CPA from the IVD, its concentration may be measured in the tissue or the solution using imaging techniques, such as CT or osmolarity tests, similar to the methods mentioned previously.

Additionally, the solution used in the restoration process may be replaced several times to maintain low levels of CPA, ensuring ideal permeation of CPA from the tissue to the solution.

Following the restoration process, the IVD is incubated in a cell culture medium for at least 24 hours and assessed for various properties, including cell viability, biomechanical properties, and more.

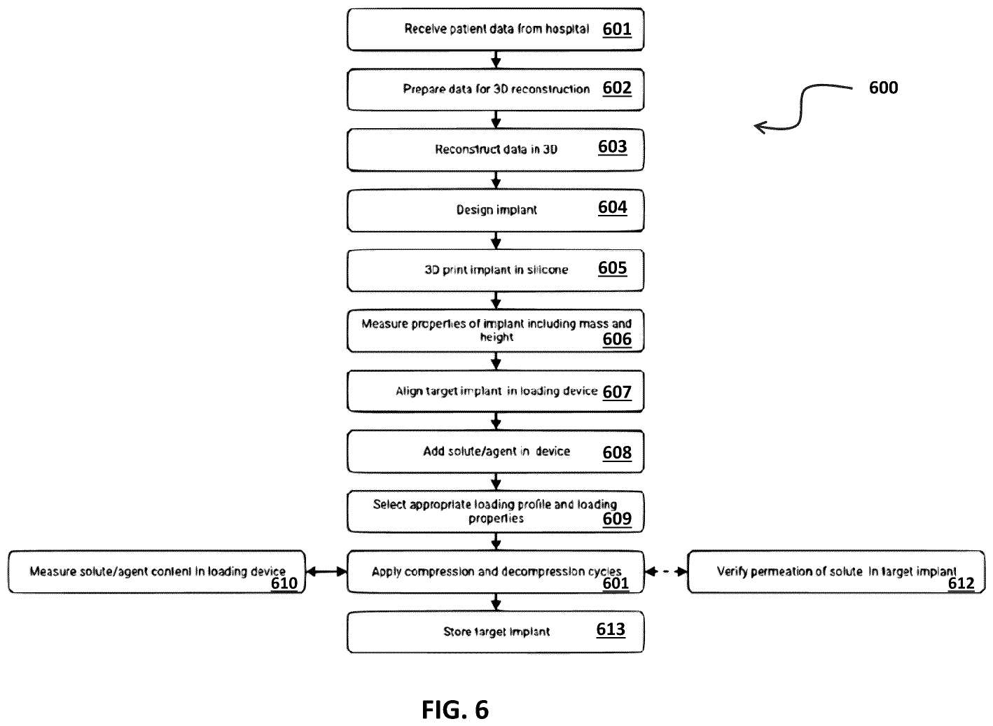

FIG. 6 is a flowchart that generally describes a cryopreservation method of tissue 600. The process may include steps for receiving tissue from a procurement facility (step 601), from a tissue source such as a human spine (e.g., spinal column 300). Steps for preparation of tissue for dissection and isolation (step 602). Dissection and separation of the tissue may be carried out under sterile conditions (step 603). Adjacent tissue that is not relevant to the target tissue may be removed (step 604). For example, the dissection of IVD 400 (e.g., see FIG. 4) may include the removal of parts of the inferior vertebral body 301 endplates (e.g., see FIG. 3) using a straight edge to maintain transport through the endplate route. The dissected tissue may include a composite tissue, an intact tissue, and parts of tissue. In some aspects of the disclosure, scaffolds such as those described herein also can be used with the method. In some aspects of the disclosure, the target tissue may be further processed before cryopreservation. For example, dissection of IVD may involve the removal of muscle and fat tissue, exposing the target IVD 400. Following the dissection steps, the tissue may be disinfected and prepared for incubation (step 603). This step may include rinsing the tissue in an antibacterial solution or in a series of disinfecting solutions. For example, solutions intended to clean and/or disinfect the tissue may be used. These solutions may contain Tyrode's balanced salt (TBSS), penicillin/streptomycin, and fungizone. In some aspects of the disclosure, the dissected tissue may be washed in a solution, such as PBS. In other aspects of the disclosure, the tissue may be wrapped with sterile gauze and soaked in a disinfecting agent, such as sodium chloride, betadine, alcohol, or other disinfecting agents. In some aspects of the disclosure, tissue properties may be collected, such as tissue height, tissue weight, tissue mechanical properties, and imaging data.

In some aspects of the disclosure, the target tissue may be kept in a solution for further processing. The solution might comprise a solvent and one or more therapeutic agents and/or restoration agents to improve, enhance, alter, and maintain tissue properties, including diffusivity, viability, mechanical properties, tissue demarcation, cell count, elasticity, porosity, ECM, chemical content, weight, height, hydration level, charge density, DNA, RNA, protein, cellular function, and tissue function. Such solutions may include growth factors, detergents, anti-coagulants, decalcifying agents, minerals, salts, vitamins, amino acids, sugars, antibiotics, antimycotic, fungicides, proteins, DNA, RNA, viruses, fetal bovine serum (FBS), EDTA, and other materials relevant to cellular and tissue biological function.

The dissected tissue may then be incubated in cell culture media at optimal conditions predetermined by the user (step 605 of FIG. 6), facilitating cell and tissue recovery. For example, the dissected IVDs may be incubated at 37° C. and 5% CO₂. Culture media may include Dulbecco's Modified Eagle Medium (DMEM), glucose, sodium pyruvate, I-glutamine, penicillin, streptomycin, gentamicin, fungizone, HEPES buffer, ascorbic acid, and Fetal Bovine Serum (FBS). In some aspects of the disclosure, methods may be used to assess tissue properties before the cryopreservation process (e.g., assessment of cell viability, measurement of mechanical properties, and quantification of physical properties like mass and height).

In some aspects of the disclosure, following the preparation of the tissue, steps 601 through 606 of FIG. 6, the target tissue may be placed in a loading apparatus 700. The loading apparatus may be configured to comprise an enclosure 701, which may be comprised of additional parts (not shown) designed to accommodate various solutions, including a saline salt solution, a cell culture medium, and a cryopreservation solution. Furthermore, the loading apparatus may be manufactured so that it maintains constant solution temperature. In some aspects of the disclosure, following the measurement of tissue properties (step 606 of FIG. 6), the tissue may be aligned within the loading apparatus (step 607 of FIG. 6). During the cryopreservation process, the target tissue is aligned with the loading mechanism 703 to provide a uniform distribution of load, which can enhance the permeation of the cryoprotective agent (CPA) within the tissue. In certain aspects of the disclosure, the loading apparatus allows for the addition of the CPA solution before receiving the tissue and/or after receiving the tissue (step 608 of FIG. 6). The CPA solution may be provided at a temperature of 4° C.

Additionally, an appropriate loading profile is determined and applied to the tissue (step 609 of FIG. 6). The application of compression may be carried out manually and/or automatically. In some aspects of the disclosure, manual compression may involve placing a weight on the first end of the compression mechanism, exerting pressure on the tissue. Alternatively, an automated system can be employed to control the loading mechanism, ensuring precise and consistent compression. In some aspects of the disclosure, the tissue is compressed until it reaches a specific height referred to as the first tissue height. Subsequently, the tissue is allowed to decompress, returning to a second height. The compression and decompression cycles may be repeated multiple times until the desired CPA permeation level is achieved. For instance, the tissue may undergo four cycles of compression and decompression until complete permeation is achieved. A user may measure the CPA content in the loading apparatus (step 610 of FIG. 6) to validate the desired permeation levels. Quality systems may be utilized to validate levels of permeation, including imaging techniques such as computed tomography (CT) and magnetic resonance imaging (MRI). For example, CT imaging of the tissue may provide quantitative information on the distribution and penetration of the CPA within the tissue. By analyzing the imaging data, the effectiveness of the cryopreservation process can be evaluated, allowing for adjustments or optimization if necessary. In some aspects of the disclosure, it may be necessary to replace the (CPA) solution between each loading step and the loading cycle.

In some representative examples of the present disclosure, the disclosed method may be utilized on an elastic biological material, such as a tendon and/or a ligament. In such aspects, applying a load to the biological material using a loading apparatus comprises using a loading apparatus with a loading mechanism to stretch the elastic biological material to facilitate distribution of a CPA, therapeutic agent, restoration agent, or a combination thereof into the elastic biological material via free-swelling. The load can then be removed to allow the biological material to be restored back to its non-stretched natural state with the agents sufficiently distributed therein.

### Restorative Method Embodiments

In other aspects of the present disclosure, the disclosed method is used to restore or sustain the health of a biological material, such as in the case of managing swelling, inflammation, and/or immune response to a graft or transplant. In such aspects, a therapeutic and/or restoration agent can be utilized in the method. In a particular aspect pertaining to using the method for facilitating transplantation, the method disclosed herein can be used to promote free swelling of the biological material so as to induce the biological material to accept a high concentration of the therapeutic agent (e.g., anti-inflammatory, immunosuppressive, and antibiotic medications). The biological material can thus serve as a functional “reservoir” for the therapeutic agent and when transplanted into a subject, the transplanted tissue itself can effectively administer a localized delivery of the infused therapeutic agent-helping to quell inflammation, enhance healing, and reducing the risk of rejection and infection post-transplantation.

Embodiments where the method is used for restoring and/or sustaining a biological material, the method enables precise control over drug concentrations in the biological material. By choosing a target drug concentration in the biological material, a “localized drug environment” surrounding the transplant site can be prepared that is highly favorable for graft acceptance and integration. For instance, the transplant biological material can be preloaded with a significantly higher concentration of immunosuppressants than is typically maintained (or generally regarded as safe) in the subject's systemic blood plasma before and after transplantation. This higher drug concentration around the transplant site can effectively dampen the immune response in that specific area, promoting successful graft integration. Additionally, in some embodiments, the release of the drug into the subject occurs in the immediate vicinity of the transplantation area, optimizing therapeutic impact where it is most needed. As the biological material integrates into the recipient's body, the therapeutic agents gradually diffuse into and equalize with the surrounding tissues, promoting graft acceptance and minimizing the risk of rejection. Importantly, since the diffusion of the drug is confined to the early stages of transplantation, and since the transplant biological material is a limited reservoir, there is minimal risk of toxicity to the subject. In the non-limiting example of targeting an immunosuppressive or anti-inflammatory environment at the transplant site, the preloading of therapeutic agents into the transplant biological material support graft survival and mitigate against the need for high levels of systemic immunosuppression.

### Applications

The disclosed method allows for long-term storage in cryoprotection and even in methods where cooling is not required (e.g., in a restoration context), ensuring the availability of biological material samples for extended periods, which may be particularly important for rare or limited biological material sources. Damage to the biological material can be minimized, preserving its biological activity, including cellular viability, structural integrity, enzymatic activity, extracellular matrix (ECM), DNA, RNA, and gene expression profiles.

Biological materials subject to method embodiments disclosed herein can serve as valuable resources for various research and development purposes, such as studying biological processes, investigating disease mechanisms, and developing new therapies. Additionally, cryopreserved and/or restored biological materials can have various applications in clinical settings, including organ transplantation, tissue engineering, regenerative medicine, and reconstructive surgeries. These applications offer potential solutions for treating various medical conditions. Cryopreservation can also play a significant role in preserving genetic resources within the tissue, including DNA, RNA, and proteins, which may have applications in genetic research, forensics, and personalized medicine.

The disclosed method may be utilized for several reasons, including but not limited to: (1) sample extraction, wherein cryopreserved biological material may contain valuable biological materials or components that need to be extracted for research, diagnostic, or therapeutic purposes; (2) allograft transplantation, wherein cryopreserved biological material can be used as allografts for transplantation into patients in need of tissue repair or replacement; (3) assessment of biological material properties, wherein thawing provides the ability to evaluate the biological material's properties, such as cellular viability, structural integrity, and biological activity.

### Loading Apparatus for Cryopreservation and/or Restoration

Described herein are embodiments of a loading apparatus used to carry out method embodiments described herein. In particular aspects of the disclosure, the loading apparatus comprises a loading mechanism for applying tension or compression to a biological material that becomes associated with the loading apparatus during use, the loading mechanism being manually or automatically operable; and an enclosure constructed with inert materials that contains the loading apparatus and that has a volume to accept a solution comprising a cryopreservation agent, therapeutic agent, restoration agent, or a combination thereof. In some aspects, the loading mechanism comprises a pair of opposing and moveable substrates that are arranged so as to position and hold the biological material between same-facing surfaces of the opposing substrates. Such embodiments can be used when static loading is utilized. In other aspects of the disclosure, the loading mechanism comprises an attachment feature that secures the biological material in a fixed position and a compression mechanism that compresses the biological material. Such embodiments can be used when dynamic loading is utilized, such as when the loading mechanism is a pneumatic piston. In yet other embodiments, the loading mechanism comprises opposing and moveable arms that are capable of holding the biological material and applying tension thereto, with particular examples utilizing a loading mechanism that is a tensioner. Such embodiments can be used when a biological material is capable of being stretched so as to facilitate free swelling as described herein, such as is the case with, for example, a tendon or ligament. Tensioning apparati can also facilitate faster decompression of tissues, such as IVD, following a compression period. In some aspects of the disclosure, the loading mechanism further comprises a locking mechanism to maintain a compression pressure or tension applied to the biological material.

In some aspects of the method, the enclosure is a bioreactor. In some such aspects, the bioreactor further comprises an integration feature that facilitates physical coupling with a cryopreservation storage unit. The enclosure can also comprise one or more ports that can be fluidly coupled to one or more tubes and/or liquid pumps that facilitate introducing the cryopreservation agent, therapeutic agent, restoration agent, or the combination thereof into and/or out of the enclosure. The enclosure typically is constructed out of an inert material. In some aspects, the inert material is a non-ferromagnetic material that avoids generating artifacts during use of the loading apparatus when the loading apparatus is used in combination with one or more medical imaging techniques.

In yet additional aspects, the loading apparatus further comprises one or more measurement features selected from a displacement sensor, a temperature sensor, a load sensor, or any combination thereof and wherein the one or more measurement features monitors parameters selected from temperature; concentration of the cryopreservation agent, therapeutic agent, restoration agent, or the combination thereof; tension; compression; and/or biological material dimensions during use of the loading apparatus.

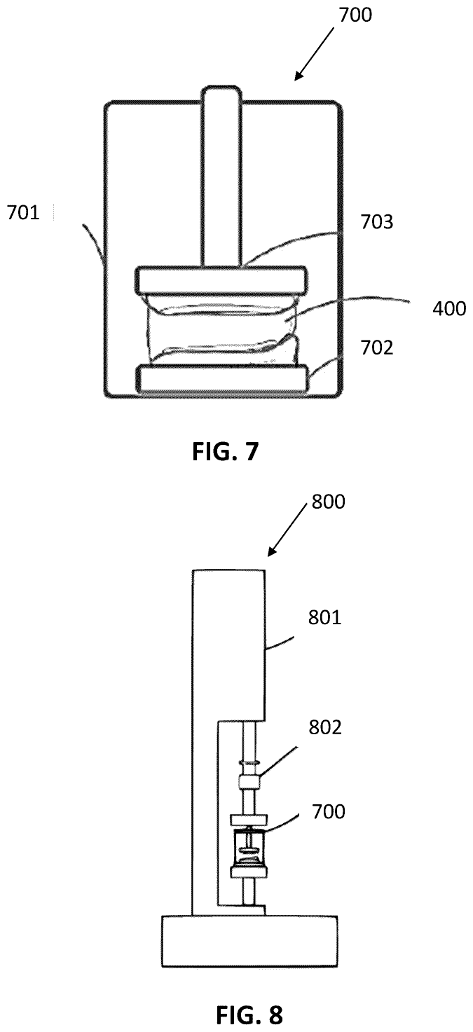

FIG. 7 is an illustration of an exemplary loading apparatus according to the present disclosure. With reference to FIG. 7, the loading apparatus 700 comprises enclosure 701 and loading mechanism 703. Loading apparatus 700 can further comprise one or more ports (not shown), one or more attachment features 702, and one or more measurement features (not shown). The enclosure is designed to have a volume that corresponds to 1× or more the volume of the target biological material, ensuring space for the biological material and cryopreservation solution. The materials used in constructing the enclosure are carefully selected to be inert. This means that the materials do not produce any chemical or biological interactions with the solutions, cells, and/or tissues contained within the enclosure. The inert nature of the materials ensures that there are no undesirable effects on the biological material or the cryopreservation process, maintaining the integrity and quality of the preserved biological material. Furthermore, the loading apparatus' components may be manufactured using non-ferromagnetic materials. For example, plastics may be used to manufacture an enclosure 701, attachment feature 702, and loading mechanism 703 to avoid generating artifacts during medical imaging techniques, such as computed tomography (CT) and magnetic imaging resonance (MRI). In some aspects of the disclosure, imaging of the loading apparatus coupled with the target biological material may be used to evaluate the cryopreservation process. For example, certain CPA types, such as dimethyl sulfoxide (DMSO), may attenuate under CT, enabling the tracking of CPA permeation levels in the target biological material. Enclosure of the loading apparatus serves as a structural element to contain and hold the biological material and other components during the cryopreservation process. The ports (not shown) are openings in the enclosure that allow for the introduction and removal of solutions as needed, providing flexibility and control over the cryopreservation procedure. The attachment feature 702 is designed to securely hold the biological material in place, ensuring proper alignment with the loading mechanism 703 and facilitating uniform distribution of load during the cryopreservation process. In some aspects of the disclosure the attachment feature may be a component of the loading mechanism 703. Furthermore, the loading apparatus 700 may incorporate measurement features and/or devices, which can include sensors or indicators that provide real-time data during the cryopreservation process. These measurement features enable the monitoring of parameters such as temperature, pressure, and biological material dimensions, allowing for precise control and evaluation of the cryopreservation process. In some aspects of the disclosure, such measurement features and/or devices may be coupled to the loading apparatus and/or any of its components.

In some aspects of the disclosure, the loading apparatus may comprise a loading mechanism 703. The compression mechanism is designed to apply controlled and uniform pressure to the target biological material 400 during the compression phase of the cryopreservation cycle. The compression mechanism may comprise mechanical components, such as springs, levers, or pneumatic systems, that allow for precise adjustment and application of the compressive force. These mechanical components are carefully calibrated to ensure consistent and reproducible compression of the biological material. The compression mechanism can be operated either manually or automatically, offering flexibility in the cryopreservation process. Manual operation allows users to make real-time adjustments based on specific requirements, while automatic operation utilizes sensors, actuators, and software algorithms to regulate the compression cycles with precision. In some aspects of the disclosure, the compression mechanism may include a locking mechanism to maintain compression when a load is not applied. In other aspects of the disclosure, the loading may be applied manually by directly applying load by the user and/or other objects.

In some aspects of the disclosure, the loading apparatus may integrate with a controller system (not shown) to enhance the cryopreservation process. The controller system includes a software package, user interface, and data input/output capabilities connected to sensors and load cells within the loading apparatus. This integration enables precise control and coordination of the compression and decompression cycles applied to the target biological material during cryopreservation. The controller system may be connected to external resources, such as a server, cloud, or computing device. This configuration allows for the transmission, storage, and analysis of relevant data related to the cryopreservation process. Data collected from the sensors and load cells can be processed and utilized by the software package to optimize the cryopreservation parameters and facilitate informed decision-making during the process. By leveraging the data obtained through the controller system, the cryopreservation process can be closely monitored and adjusted in real time. This dynamic control ensures consistency, reproducibility, and precision in the application of compression and decompression cycles, resulting in enhanced biological material preservation outcomes.

In some aspects of the disclosure, the loading apparatus may be coupled with other devices that receive, process, compile, store, and send data. In some aspects of the disclosure, the loading apparatus may be coupled with separate devices (e.g., a smartphone, a tablet, a computer, a solution pump, a load cell, a displacement sensor) and/or storage medium (e.g., a flash drive, a memory card). In some aspects of the disclosure, the loading apparatus may be thoughtfully designed to be compatible with a cryopreservation storage unit, presenting numerous benefits that streamline and enhance the cryopreservation workflow. This configuration of the loading apparatus ensures seamless and direct transfer of the preserved biological material from the loading apparatus to the designated cryopreservation storage unit without any intermediary steps or repositioning. This seamless transfer minimizes the risk of damage and preserves the structural and functional integrity of the sample, mitigating potential risks associated with multiple transfers.

In some aspects of the disclosure, a loading system can be employed to facilitate, control, assist the loading mechanism, ensuring precise and consistent compression. As shown in FIG. 8, this system 800 may comprise frame 801 and a loading mechanism 802. In some aspects of the disclosure, sensors, measurement devices, and a solution handling apparatus may be integrated and/or coupled to the loading system 800.

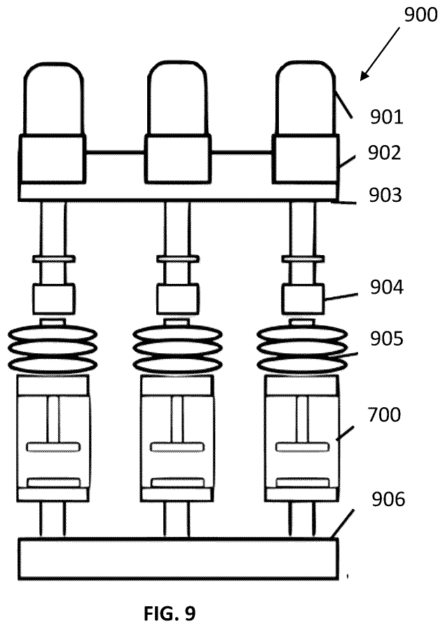

FIG. 9 is an illustration of a loading frame system (e.g., compression frame system 900). In this case a bioreactor is used as an example of a compression frame system. In some aspects of the disclosure, a plurality of loading apparatuses 700 can be used as part of a bioreactor system. In some aspects of the disclosure, each apparatus may be run independently of the other loading apparatus in a bioreactor system. Yet in another aspect of the disclosure, all apparatus may run simultaneously. Each apparatus may be designed to support one type of biological material under controlled conditions. These conditions include precise regulation of temperature, CO₂ and O₂, circulation of nutrients, and the option for dynamic or static loading.

The loading apparatus and system may utilize a compression mechanism the like of a pneumatic piston 901 to apply controlled mechanical loads on the biological material. The pneumatic piston may exert pressure directly on the biological material. In some aspects of the disclosure, the pneumatic system is connected to an air pressure regulator 902 which adjusts the amount of air used to control loading. The load applied may be controlled manually and/or automatically using a computing system. In some aspects of the disclosure, such computing systems may determine, supply, and control the voltage input sent to the air regulator and/or loading mechanism control unit. In other aspects of the disclosure, the bioreactor system may apply pressure to the loading mechanism of the compression apparatus, which subsequently applies the load onto the biological material. In other aspects of the disclosure, the load may be applied to a component of the compression frame system, such as a compression feature 804. In some aspects of the disclosure, the various components and features of the compression frame system may be fastened to a frame or gear 903.

In some aspects of the disclosure, the bioreactor system is coupled with sensing devices and apparatus to intermittently, continuously, sparingly monitor the biological material properties, such as height changes, load, volume, and temperature. Additionally, such features may be measured and monitored using a displacement sensor, an electrode, a load cell 906, a spectrophotometer to accurately measure biological material properties during the cryopreservation process. In some aspects of the disclosure, such measurement devices and apparatus may be coupled to each compression apparatus. Additionally, a feature 905 such as a rubber sleeve may be used to cover the interface between the apparatus and the compression frame system to prevent contamination of the environment inside the compression apparatus.

In some aspects of the disclosure, the bioreactor system may be coupled with tubes and liquid pumps to control flow of cryoprotective agents (CPAs) to each and/or all compression apparatus.

### System

Also disclosed herein is a system that can be used in method embodiments described herein. In some aspects of the disclosure, the system comprises a loading apparatus as described herein and a controller system in electrical communication with the loading apparatus, wherein the controller system regulates a loading cycle applied to the biological material. The controller system typically is electronically coupled with the one or more sensing devices and/or the loading mechanism of the loading apparatus, and is operable to control system parameters and facilitate real-time adjustments during use of the system.

In some aspects, the system can further comprise: (i) one or more measurement features that monitors parameters selected from temperature, concentration, tension, compression, and/or biological material dimensions during use of the system; (ii) one or more imaging data modalities, selected from magnetic resonance imaging (MRI) data, computed tomography (CT) data, positron emission tomography (PET) data, ultrasound data, X-ray data, optical scanning data, microwave imaging data, radar ultrasound data, photogrammetry data, elastography data, and stereo-imaging data, for evaluating distribution and attenuation of the cryopreservation agent, therapeutic agent, restoration agent, or the combination thereof within the biological material; (iii) one or more additional loading apparatuses and one or more additional enclosures, wherein each of the one or more additional loading apparatuses independently is contained within one of the one or more additional enclosures; (iv) a regulation component for regulating temperature, a CO₂ level, an O₂ level, and/or circulation of nutrients within the enclosure and the one or more additional enclosures; (v) one or more sensing devices for monitoring biological material properties selected from tensile changes, height changes, volume, and/or temperature, wherein the one or more sensing devices is physically associated with or coupled to the biological material, the loading apparatus (and/or the one or more additional loading apparatus), the enclosure (and/or the one or more additional enclosures), or a combination thereof; (vi) a plurality of tubes, ports, and liquid pumps that are independently physically or fluidly coupled to one or more of the enclosure (and/or the one or more additional enclosures) and that facilitate delivery of the cryopreservation agent, therapeutic agent, restoration agent, or the combination thereof into and/or out of the system; (vii) a data acquisition unit that is electronically coupled to the system; or (viii) any combination of two or more of (i)-(vii).

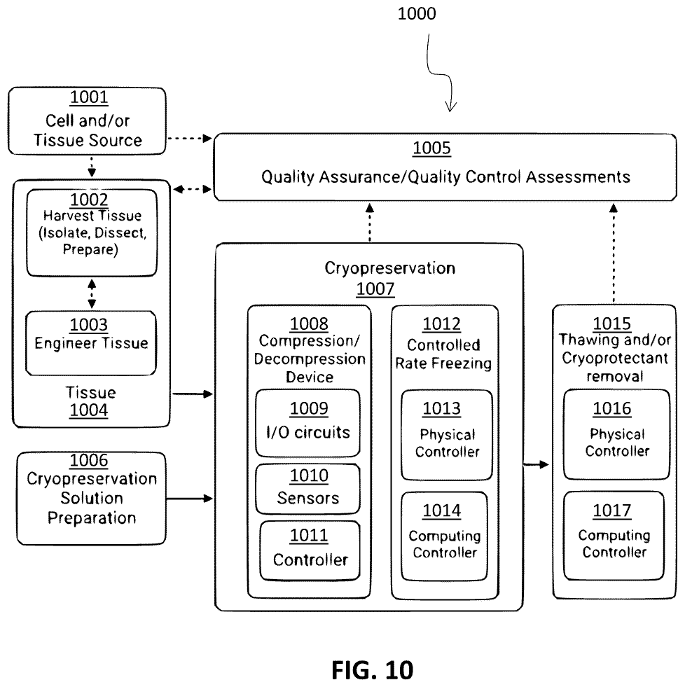

FIG. 10 illustrates a high-level block diagram of system 1000, in accordance with at least one embodiment of the present disclosure. With reference to FIG. 10, cells and/or tissue/s may or may not be obtained from a source 1001, herein referred to as “source,” including, but not limited to, any individual or combination of the following: living or deceased human donors, living or deceased mammalian animal donors, plants, bacteria, or fungi. An engineered tissue could be derived from synthetic components and not require an organic source. The source may or may not undergo quality assurance/quality control assessment as described below. The cell and/or tissue harvest 1002 may or may not require additional isolation, dissection, or other preparation. Cells and/or tissues from the source/s may or may not be used in the creation of an engineered tissue 1003. There may or may not be a combination of harvested and engineered tissue comprising the ultimate tissue for cryopreservation 1004, herein referred to as “tissue.” The tissue may or may not undergo quality control/quality assurance assessment 1005 at this point in the process or later in the process. These quality assessments may include but are not limited to the following: (1) evaluation of the medical record of the source; (2) evaluation of source medical images including, but not limited to, imaging modalities, such as magnetic resonance imaging (MRI), computed tomography (CT), positron emission tomography (PET), ultrasound, X-ray, optical scanning, photography, and elastography; (3) evaluation of tissue biochemistry; (4) evaluation of cell viability and phenotype; (5) evaluation of tissue histology; (6) evaluation of soluble biomarkers; (7) assessment of tissue gross morphology; (8) assessment of CPA permeation of the tissue through assay measurement or imaging via one of the following methods: magnetic resonance imaging (MRI), computed tomography (CT), positron emission tomography (PET), ultrasound, X-ray, optical scanning, photography, microwave imaging, radar ultrasound, and elastography. In particular, the assessment of CPA permeation via noninvasive imaging methods is of great benefit as it allows for further tissue processing, prior to cryopreservation, if necessary.

A cryopreservation solution (CPS) 1006, containing one or more types of CPA and one or more buffers, is prepared for addition to the tissue. The CPS may include but is not limited to dimethyl sulfoxide (DMSO), ethylene glycol (EG), and propylene glycol (PG).

The CPS is then added to a container with the tissue for the cryopreservation 1007 process. For cryopreservation to be successful, the tissue must be completely permeated by the CPS and frozen before the CPS damages the cells therein. The method specified here utilizes a loading apparatus 1008 to compress and/or decompress tissue. The loading apparatus may be a simple manual load application or contain the following components individually or in combination: I/O circuits 1009 to connect with sensors 1010 and/or an internal or external controller 1011. The sensor could utilize mechanical mechanisms to indicate heights, pressures, or other physiochemical properties in the system, for instance, a thermometer, a spring-measured force gauge, or a micrometer. The sensor could also be more sophisticated, such as an analog load cell. The controller can likewise encompass a range of technology from manually turning a screw to applying a load to a computerized feedback or feedforward control system. Several embodiments of these apparatus are described in FIGS. 7, 8, and 9.

Following or overlapping with the compression and decompression of the tissue is a controlled rate of cooling (e.g., freezing 112) to a predetermined temperature.

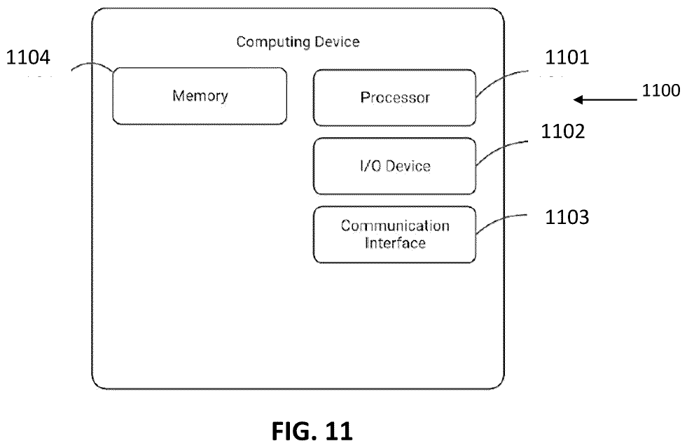

FIG. 11 depicts a block diagram of a computing device 1100 configured to facilitate data processing, analysis, and control during the cryopreservation process. The computing device acts as the central unit that manages and coordinates various aspects of the cryopreservation procedure, ensuring precise control, real-time monitoring, and optimization of the parameters involved. While a single computing device is schematically depicted, the systems described herein may include a plurality (e.g., two, three, four, five, or more) computing devices. In some aspects of the disclosure, the cryopreservation system incorporates a full-stack technology within the computing device and other devices, enabling seamless integration and real-time processing, analysis, and interpretation of data from various sensors and measurement features. The computing device's full-stack technology encompasses raw data collection from a diverse set of devices, including sensors within the loading apparatus, parameters of solutions utilized during the cryopreservation process, external servers, and databases, collecting data on biological material response, compression, decompression, CPA permeation, and critical parameters. Data storage capabilities efficiently archive and manage substantial volumes of cryopreservation data, facilitating data analysis and data-driven decision-making. In some aspects of the disclosure, the computing device may be configured to integrate with load cells and displacement sensors to provide precise and continuous measurement of mechanical forces and biological material deformations, ensuring tailored optimization of the cryopreservation process for each biological material type. Advanced compression and decompression mechanisms, combined with real-time data processing, foster uniform CPA distribution within the biological material.

Generally, the computing devices described herein may comprise a controller comprising a processor 1101 (e.g., CPU) and a memory 1104 (which can include one or more computer-readable storage mediums). The processor may incorporate data received from memory and user input to control one or more components of the system (e.g., loading apparatus, load cell, devices, and other computing device). The memory may further store instructions to cause the processor to execute modules, processes, and/or functions associated with the methods described herein. As used herein, a computing device may refer to any of the sensors, controller, and computing controller as depicted in FIG. 10. In some aspects of the disclosure, the memory and the processor may be implemented on a single chip. In other aspects of the disclosure, they can be implemented on separate chips.

The computing device may be configured to receive and process user input to the computing device and data from the cryopreservation process. The computing device may be configured to receive, compile, store, and access data. In some aspects of the disclosure, the computing device may be configured to access and/or receive data from different sources. The computing device may be configured to receive data directly input by a user and/or it may be configured to receive data from separate devices (e.g., a smartphone, a tablet, a computer, a load cell) and/or from a storage medium (e.g., a flash drive, a memory card). The computing device may receive the data through a network connection, as discussed in more detail herein, or through a physical connection with the device or storage medium (e.g., through a Universal Serial Bus (USB) or any other type of port). The computing device may include any of a variety of devices, such as a cellular telephone (e.g., smartphone), tablet computer, laptop computer, desktop computer, portable media player, wearable digital device (e.g., digital glasses, wristband, wristwatch, brooch, armbands, virtual reality/augmented reality headset, jewelry (e.g., bracelet, necklace, ring)), television, set-top box (e.g., cable box, video player, video streaming device), gaming system, or the like.

The processor may be any suitable processing device configured to run and/or execute a set of instructions or code and may include one or more data processors, image processors, graphics processing units, physics processing units, digital signal processors, and/or central processing units. The processor may be, for example, a general-purpose processor, Field Programmable Gate Array (FPGA), an Application Specific Integrated Circuit (ASIC), and/or the like. The processor may be configured to run and/or execute application processes and/or other modules, processes, and/or functions associated with the system and/or a network associated therewith. The underlying device technologies may be provided in a variety of component types (e.g., metal-oxide-semiconductor field-effect transistor (MOSFET) technologies like complementary metal-oxide semiconductor (CMOS), bipolar technologies like emitter-coupled logic (ECL), polymer technologies (e.g., silicon-conjugated polymer and metal-conjugated polymer-metal structures), mixed analog and digital, and/or the like.

In some aspects of the disclosure, the memory may include a database (not shown) and may be, for example, a random-access memory (RAM), a memory buffer, a hard drive, an erasable programmable read-only memory (EPROM), an electrically erasable read-only memory (EEPROM), a read-only memory (ROM), Flash memory, and the like. The memory may store instructions to cause the processor to execute modules, processes, and/or functions associated with the communication device, such as measurement data processing, measurement device control, communication, and/or device settings. Some aspects of the disclosure described herein relate to a computer storage product with a non-transitory computer-readable medium (also may be referred to as a non-transitory processor-readable medium) having instructions or computer code thereon for performing various computer-implemented operations. The computer-readable medium (or processor-readable medium) is non-transitory in the sense that it does not include transitory propagating signals per se (e.g., a propagating electromagnetic wave carrying information on a transmission medium such as space or a cable). The media and computer code (also may be referred to as code or algorithm) may be those designed and constructed for a specific purpose or purposes.

Examples of non-transitory computer-readable media include but are not limited to, magnetic storage media such as hard disks, floppy disks, and magnetic tape; optical storage media such as Compact Disc/Digital Video Discs (CD/DVDs); Compact Disc-Read Only Memories (CD-ROMs), and holographic devices; magneto-optical storage media such as optical disks; solid-state storage devices such as a solid-state drive (SSD) and a solid-state hybrid drive (SSHD); carrier wave signal processing modules; and hardware devices that are specially configured to store and execute program code, such as Application-Specific Integrated Circuits (ASICs), Programmable Logic Devices (PLDs), Read-Only Memory (ROM), and Random-Access Memory (RAM) devices. Other aspects of the disclosure described herein relate to a computer program product, which may include, for example, the instructions and/or computer code disclosed herein.

The systems, devices, and/or methods described herein may be performed by software (executed on hardware), hardware, or a combination thereof. Hardware modules may include, for example, a general-purpose processor (or microprocessor or microcontroller), a field programmable gate array (FPGA), and/or an application-specific integrated circuit (ASIC). Software modules (executed on hardware) may be expressed in a variety of software languages (e.g., computer code), including C, C++, Java®, Python, Ruby, Visual Basic®, and/or other object-oriented, procedural, or other programming language and development tools. Examples of computer code include but are not limited to, micro-code or micro-instructions, machine instructions, such as produced by a compiler, code used to produce a web service, and files containing higher-level instructions that are executed by a computer using an interpreter. Additional examples of computer code include but are not limited to, control signals, encrypted code, and compressed code.

In some aspects of the disclosure, the computing device may further comprise a communication interface configured to permit a user to control one or more of the devices of the system. The communication interface may comprise a network interface configured to connect the computing device to another system (e.g., Internet, remote server, database) by wired or wireless connection. In some aspects of the disclosure, the computing device may be in communication with other devices via one or more wired and/or wireless networks. In some aspects of the disclosure, the network interface may comprise a radiofrequency receiver, transmitter, and/or optical (e.g., infrared) receiver and transmitter configured to communicate with one or more devices and/or networks. The communication interface may communicate by wires and/or wirelessly with one or more of the loading apparatus, networks, solutions devices, servers, and other computing devices.

The network interface may comprise RF circuitry that may receive and send RF signals. The RF circuitry may convert electrical signals to/from electromagnetic signals and communicate with communications networks and other communications devices via the electromagnetic signals. The RF circuitry may comprise well-known circuitry for performing these functions, including but not limited to an antenna system, an RF transceiver, one or more amplifiers, a tuner, one or more oscillators, a digital signal processor, a CODEC chipset, a subscriber identity module (SIM) card, memory, and so forth.

Wireless communication through any of the computing and measurement devices may use any of plurality of communication standards, protocols and technologies, including but not limited to, Global System for Mobile Communications (GSM), Enhanced Data GSM Environment (EDGE), high-speed downlink packet access (HSDPA), high-speed uplink packet access (HSUPA), Evolution, Data-Only (EV-DO), HSPA, HSPA+, Dual-Cell HSPA (DC-HSPDA), long term evolution (LTE), near field communication (NFC), wideband code division multiple access (W-CDMA), code division multiple access (CDMA), time division multiple access (TDMA), Bluetooth, Wireless Fidelity (WiFi) (e.g., IEEE 802.11a, IEEE 802.11b, IEEE 802.11g, IEEE 802.11n, and the like), voice over Internet Protocol (VOIP), Wi-MAX, a protocol for e-mail (e.g., Internet message access protocol (IMAP) and/or post office protocol (POP)), instant messaging (e.g., extensible messaging and presence protocol (XMPP), Session Initiation Protocol for Instant Messaging and Presence Leveraging Extensions (SIMPLE), Instant Messaging and Presence Service (IMPS)), and/or Short Message Service (SMS), or any other suitable communication protocol. In some aspects of the disclosure, the devices herein may directly communicate with each other without transmitting data through a network (e.g., through NFC, Bluetooth, WiFi, RFID, and the like).

The input/output (I/O) device (e.g., 1102 of FIG. 11) may comprise a user interface (e.g., 1103 of FIG. 11) configured to permit a user to control the computing device. The communication interface may permit a user to interact with and/or control a computing device directly and/or remotely. For example, the input/output device may include an input device for a user to input commands and an output device for a user to receive output. An output device of the I/O device may output compression data and biological material deformation distance and may comprise one or more of a display device and audio device. Furthermore, data analysis may be displayed by the output device (e.g., display). In some aspects of the disclosure, an output device may comprise a display device including at least one of a light emitting diode (LED), liquid crystal display (LCD), electroluminescent display (ELD), plasma display panel (PDP), thin film transistor (TFT), organic light-emitting diodes (OLED), electronic paper/e-ink display, laser display, and/or holographic display.

An audio device may audibly output user data, data analysis, system data, alarms, and/or notifications. For example, the audio device may output an audible alarm when the compression and/or decompression cycle is complete. In some aspects of the disclosure, an audio device may comprise at least one of a speaker, piezoelectric audio device, magnetostrictive speaker, and/or digital speaker. In some aspects of the disclosure, a user may communicate with other users using the audio device and a communication channel. For example, a user may form an audio communication channel (e.g., VoIP call) with a remote health care professional.

In some aspects of the disclosure, the I/O device may comprise an input device (e.g., touch screen) and output device (e.g., display device) and be configured to receive input data from one or more of the loading apparatus, network, solution(s) device, server, and other computing device. For example, user control of an input device (e.g., keyboard, buttons, touch screen) may be received by the I/O device and may then be processed by processor and memory for the I/O device to output a control signal to the solution device. Some aspects of the disclosure of an input device may comprise at least one switch configured to generate a control signal. For example, an input device may comprise a touch surface for a user to provide input (e.g., finger contact to the touch surface) corresponding to a control signal. An input device comprising a touch surface may be configured to detect contact and movement on the touch surface using any of a plurality of touch sensitivity technologies, including capacitive, resistive, infrared, optical imaging, dispersive signal, acoustic pulse recognition, and surface acoustic wave technologies. In aspects of the disclosure of an input device comprising at least one switch, a switch may comprise, for example, at least one of a button (e.g., hard key, soft key), touch surface, keyboard, analog stick (e.g., joystick), directional pad, mouse, trackball, jog dial, step switch, rocker switch, pointer device (e.g., stylus), motion sensor, image sensor, and microphone. A motion sensor may receive user movement data from an optical sensor and classify a user gesture as a control signal. A microphone may receive audio data and recognize a user voice as a control signal.

A haptic device may be incorporated into I/O device to provide additional sensory output (e.g., force feedback) to the user. For example, a haptic device may generate a tactile response (e.g., vibration) to confirm user input to an input device (e.g., touch surface). As another example, haptic feedback may notify that the computing device overrides user input.

### Industrial Production Environment for Compression-Mediated Cryopreservation

In an industrial production environment, a scalable and efficient cryopreservation system can be established by organizing banks of a loading apparatus or system [700, 800]. Each bank comprises multiple apparatus units designed to handle the preservation of specific types of biological materials, or to serve as backups for other banks during maintenance periods. The centralized control of these banks facilitates seamless coordination and optimal utilization of resources. To achieve this, each loading apparatus unit and each bank of units is assigned unique network addresses within a dedicated network infrastructure. This network facilitates transmitting control data from a central processing system to the individual units, and collecting sensing data from each loading apparatus (wherein each loading apparatus is mediated by a computing device [1100]) into a central processing system. The central processing system acts as the nerve center, managing and monitoring the entire cryopreservation production process, and presenting operating dashboards and control interfaces for use by individual human users (equipment operators and production managers) responsible for the production process.

The central control system coordinates the activities of all loading apparatus units across the banks. It can schedule and prioritize tasks based on biological material type, production volume, and equipment availability, ensuring efficient resource allocation and minimal downtime. This centralized approach allows for a streamlined workflow, facilitating continuous operation even when one bank is temporarily offline for maintenance or repair.

The central processing system also receives real-time sensing data from each loading apparatus, including information on load profile, temperature, pressure, biological material dimensions, solute concentrations, byproduct concentrations, CPA permeation levels, liquid flow rates, air flow rates, weight, and other metrics (e.g., pH and alkalinity). The data are used for monitoring the cryopreservation process, detecting anomalies, and making necessary adjustments to optimize biological material preservation outcomes.

The scalability of the cryopreservation system enables production volume to be easily adjusted to meet demand. Additional banks of loading apparatus can be added as needed, and the central control system can efficiently manage the increasing complexity and scale of the production process.

With this production system running the disclosed method at scale, various biological materials can be preserved effectively and consistently for medical applications, research, and other uses. The centralized control and networked infrastructure ensure that the entire cryopreservation process is well-coordinated, data-driven, and easily scalable to meet the demands of an industrial-scale production environment.

While the above description is made with reference to a cryopreservation-specific method, the above described steps and/or components can be adapted for use in restoring a biological material using a therapeutic agent and/or a restoration agent.

### Overview of Several Embodiments

Disclosed herein are embodiments of an apparatus, comprising: a loading mechanism for applying tension or compression to a biological material that becomes associated with the loading apparatus during use, the loading mechanism being manually or automatically operable; and an enclosure constructed with inert materials that contains the loading apparatus and that has a volume to accept a solution comprising a cryopreservation agent, therapeutic agent, restoration agent, or a combination thereof.

In any or all of the above embodiments, (i) the loading mechanism comprises a pair of opposing and moveable substrates that are arranged so as to position and hold the biological material between same-facing surfaces of the opposing substrates; (ii) the loading mechanism comprises an attachment feature that secures the biological material in a fixed position and a compression mechanism that compresses the biological material; or (iii) the loading mechanism comprises opposing and moveable arms that are capable of holding the biological material and applying tension thereto.

In any or all of the above embodiments, the loading mechanism further comprises a locking mechanism to maintain a compression pressure or tension applied to the biological material.

In any or all of the above embodiments, the loading mechanism is a pneumatic piston or a tensioner.

In any or all of the above embodiments, the enclosure is a bioreactor and further comprises an integration feature that facilitates physical coupling with a cryopreservation storage unit.

In any or all of the above embodiments, the loading apparatus further comprises one or more measurement features selected from a displacement sensor, a temperature sensor, a load sensor, or any combination thereof and wherein the one or more measurement features monitors parameters selected from temperature; concentration of the cryopreservation agent, therapeutic agent, restoration agent, or the combination thereof; tension; compression; and/or biological material dimensions during use of the loading apparatus.

In any or all of the above embodiments, the enclosure comprises one or more ports that can be fluidly coupled to one or more tubes and/or liquid pumps that facilitate introducing the cryopreservation agent, therapeutic agent, restoration agent, or the combination thereof into and/or out of the enclosure.

In any or all of the above embodiments, the inert material is a non-ferromagnetic material that avoids generating artifacts during use of the loading apparatus when the loading apparatus is used in combination with one or more medical imaging techniques.

Also disclosed herein are embodiments of a system, comprising: the loading apparatus according to any or all of the above embodiments; and a controller system in electrical communication with the loading apparatus, wherein the controller system regulates a loading cycle applied to the biological material.

In any or all of the above embodiments, the system further comprises: (i) one or more measurement features that monitors parameters selected from temperature, concentration, tension, compression, and/or biological material dimensions during use of the system; (ii) one or more imaging data modalities that provides magnetic resonance imaging (MRI) data, computed tomography (CT) data, positron emission tomography (PET) data, ultrasound data, X-ray data, optical scanning data, microwave imaging data, radar ultrasound data, photogrammetry data, elastography data, stereo-imaging data, or a combination thereof for evaluating distribution and attenuation of the cryopreservation agent, therapeutic agent, restoration agent, or the combination thereof within the biological material; (iii) one or more additional loading apparatuses and one or more additional enclosures, wherein each of the one or more additional loading apparatuses independently is contained within one of the one or more additional enclosures; (iv) a regulation component for regulating temperature, a CO₂ level, an O₂ level, and/or circulation of nutrients within the enclosure and the one or more additional enclosures; (v) one or more sensing devices for monitoring biological material properties selected from tensile changes, height changes, volume, and/or temperature, wherein the one or more sensing devices is physically associated with or coupled to the biological material, the loading apparatus (and/or the one or more additional loading apparatus), the enclosure (and/or the one or more additional enclosures), or a combination thereof; (vi) a plurality of tubes, ports, and liquid pumps that are independently physically or fluidly coupled to one or more of the enclosure (and/or the one or more additional enclosures) and that facilitate delivery of the cryopreservation agent, therapeutic agent, restoration agent, or the combination thereof into and/or out of the system; (vii) a data acquisition unit that is electronically coupled to the system; or (viii) any combination of two or more of (i)-(vii).

In any or all of the above embodiments, the controller system is electronically coupled with the one or more sensing devices and/or the loading mechanism of the loading apparatus, and wherein the controller system is operable to control system parameters and facilitate real-time adjustments during use of the system.

Also disclosed herein are embodiments of a method, comprising: exposing a biological material to a cryopreservation agent, therapeutic agent, restoration agent, or a combination thereof; applying a load to the biological material using a loading apparatus; and removing the load from the biological material; wherein applying or removing the load facilitates distribution of the cryopreservation agent, therapeutic agent, restoration agent, or the combination thereof within the biological material.

In any or all of the above embodiments, applying the load to the biological material comprises compressing the biological material using the loading apparatus and removing the load facilitates distribution of the cryopreservation agent, therapeutic agent, restoration agent, or the combination thereof within the biological material by allowing the biological material to decompress back to its natural state.

In any or all of the above embodiments, applying the load facilitates distribution of the cryopreservation agent, therapeutic agent, restoration agent, or the combination thereof within the biological material by stretching the biological material and removing the load comprises releasing the biological material from the stretched position.

In any or all of the above embodiments, the cryopreservation agent is selected from dimethylsulfoxide (DMSO), ethylene glycol, propylene glycol, butanediol, cyclohexanediol, diethylene glycol, 3-methoxy-1,2 propanediol, glycerol, formamide, acetamide, propionamide, lactamide, malonamide, dimethylformamide, methylacetamide, dimethylacetamide, betaine, monoacetin, triacetin, ice recrystallization inhibitors, antioxidants, apoptosis inhibitors, metformin, alginate, egg yolk, polyethylene glycol (PEG), polyvinylpyrrolidone (PVP), raffinose, sucrose, trehalose, ethanol, ficoll, polyvinyl alcohol (PVA), methylsulfonylmethane (MSM), polyethyleneimine (PEI), hydroxypropyl cellulose, dextran, or combinations thereof.

In any or all of the above embodiments, the therapeutic agent may be selected from anti-inflammatory agents, immunosuppressive agents, immunomodulatory agents, vaccines, chondrogenic differentiation agents, cancer-specific inhibitors, gene therapies, anti-aging, anti-fibrotic agents, cell-survival signaling agents, pain management agents, osteogenesis agents, pan-caspase inhibitors, senolytics/senotherapeutics, antiapoptotic agents, or antibiotic drugs; and/or the restoration agent can be a nutrient.

In any or all of the above embodiments, the biological material is selected from living tissue, donor tissue, tissue composites, scaffolds, artificially manufactured tissues, biocompatible materials, or combinations thereof.

In any or all of the above embodiments, the method further comprises obtaining a signal and quantifying an amount of the cryopreservation agent, therapeutic agent, restoration agent, or combination thereof that has permeated the biological material using one or more imaging data modalities selected from MRI data, CT data, PET data, ultrasound data, X-ray data, optical scanning data, microwave imaging data, radar ultrasound data, photogrammetry data, elastography data, or stereo-imaging data.

In any or all of the above embodiments, the cryopreservation agent is used and the method further comprises: (i) cooling the biological material to a predetermined temperature to provide a cryopreserved biological material; (ii) storing the cryopreserved biological material in a cryogenic unit that maintains the cryopreserved biological material at a cryopreservation temperature; or (iii) a combination of (i) and (ii).

In any or all of the above embodiments, the method further comprises: (i) thawing the cryopreserved biological material to provide a thawed biological material; (ii) exposing the thawed biological material to a restoration solution comprising a restoration agent; (iii) applying a load to the thawed biological material in the presence of the restoration solution using a loading apparatus; and (iv) removing the load from the thawed biological material; wherein applying or removing the load facilitates removing the cryopreservation agent from the thawed biological material and also facilitates uniform distribution of the restoration solution within the thawed biological material.

In any or all of the above embodiments, the biological material is an intervertebral disc.

In any or all of the above embodiments, the method uses the cryopreservation agent and the method facilitates an increase in an amount of the cryopreservation agent that is distributed throughout the biological material within a time period where minimal to no cell death occurs in the biological material.

Disclosed herein are embodiments of a system for cryopreservation for preserving biological materials, tissues, and composites, comprising: an apparatus for applying controlled and uniform pressure to biological materials or tissue during the cryopreservation process; an enclosure constructed with inert materials, securely holding biological materials or tissue and containing a cryopreservation solution during the cryopreservation process; and a controller system in communication with the compression/decompression apparatus, regulating the compression and decompression cycles applied to the tissue

In any or all of the above embodiments, the apparatus comprises: a loading mechanism for applying the controlled pressure to the tissue, the loading mechanism being operable manually or automatically; and one or more measurement features for monitoring parameters, including temperature, concentration, load, flow rate, and dimensions during the cryopreservation process.

In any or all of the above embodiments, the controller system is coupled with sensors and load cells within the apparatus to optimize the cryopreservation parameters and facilitate real-time adjustments during the cryopreservation process.

In any or all of the above embodiments, the cryopreservation system further comprising one or more imaging data modalities, selected from the group consisting of magnetic resonance imaging (MRI) data, computed tomography (CT) data, positron emission tomography (PET) data, ultrasound data, X-ray data, optical scanning data, microwave imaging data, radar ultrasound data, photogrammetry data, elastography data, and stereo-imaging data, for evaluating the distribution and attenuation of cryoprotective agents (CPAs) within the tissue.

Also disclosed are embodiments of a method to improve transport of solutes within sub-perfusable tissues, comprising the steps preparing the tissue for cryopreservation, including dissection and separation under sterile conditions and removal of adjacent tissue not relevant to the target tissue; permeating the tissue with a cryopreservation solution; applying controlled and uniform pressure to the tissue using an apparatus to facilitate uniform distribution of the cryopreservation solution within the tissue; and cooling the tissue to a predetermined temperature; and storing the cryopreserved tissue in specialized cryogenic units.

In any or all of the above embodiments, the method further comprises evaluating cryopreservation parameters using one or more imaging data modalities consisting of MRI data, CT data, PET data, ultrasound data, X-ray data, optical scanning data, microwave imaging data, radar ultrasound data, photogrammetry data, elastography data, and stereo-imaging data.

In any or all of the above embodiments, the step of permeating the tissue with a cryopreservation solution comprises applying a controlled compression and decompression cycle using a loading mechanism within the compression/decompression apparatus.

In any or all of the above embodiments, the post-thaw step to remove CPA comprises using the apparatus to apply controlled and gradual decompression.

In any or all of the above embodiments, exposure of tissue to CPA may be minimized using compression of tissue in a low-CPA and/or free-CPA solution.

In any or all of the above embodiments, decompression, also referred to as free-swelling of tissue, is done in a medium containing CPA.

In any or all of the above embodiments, compression and decompression are performed on biological materials including, but not limited to, living tissue, donor tissue, tissue composites, scaffolds, artificially manufactured tissues, and biocompatible materials.

In any or all of the above embodiments, compression and decompression is used to improve transport of soluble compounds within sub-perfusable tissues and biological materials.

Also disclosed are embodiments of an apparatus for cryopreservation of tissue, comprising: a loading mechanism operable to apply controlled and uniform pressure to the tissue during the cryopreservation process; an enclosure constructed with inert materials to securely hold the tissue and contain a cryopreservation solution during the cryopreservation process; and one or more measurement features for monitoring parameters, including temperature, pressure, and tissue dimensions during the cryopreservation process; ports that allow addition, replacement, and removal of solution; ports and tubes coupled to a liquid pump.

In any or all of the above embodiments, the apparatus further comprising a controller system in communication with the loading mechanism, regulating the compression and decompression cycles applied to the tissue.

In any or all of the above embodiments, the loading mechanism is designed to be operable either manually or automatically.

In any or all of the above embodiments, the enclosure is designed to have a volume that corresponds to at least 1× the volume of the tissue.

In any or all of the above embodiments, the one or more measurement features comprise displacement sensors, temperature sensors, and load cells.

In any or all of the above embodiments, the one or more measurement features are coupled with a controller system, allows the apparatus to provide real-time data during the cryopreservation process.

In any or all of the above embodiments, the loading mechanism is further equipped with a locking mechanism to maintain compression when a load is not applied.

In any or all of the above embodiments, the loading mechanism is calibrated to ensure consistent and reproducible compression or tension of the tissue.

In any or all of the above embodiments, the enclosure is designed with ports allowing for the introduction and removal of solutions during the cryopreservation process.

In any or all of the above embodiments, the ports are coupled to tubes.

In any or all of the above embodiments, the enclosure is constructed using non-ferromagnetic materials to avoid generating artifacts during medical imaging techniques.

In any or all of the above embodiments, the apparatus further comprises an integration feature that enables coupling with a cryopreservation storage unit to directly transfer the cryopreserved tissue.

In any or all of the above embodiments, the ports are connected to tubes to a liquid pump.

In any or all of the above embodiments, the pump is a peristaltic pump that provides a flow of the cryopreservation solution within the enclosure.

In any or all of the above embodiments, the apparatus further comprises a user interface allowing an operator to control and adjust compression parameters and solution flow rates.

In any or all of the above embodiments, the apparatus is coupled to a compression frame system such as a bioreactor.

Also disclosed are embodiments of a compression and/or tension frame system, comprising a plurality of compression and/or tension apparatuses, each designed to support one tissue or biological material; a component for precise regulation of temperature, CO₂, O₂, circulation of nutrients within each compression and/or tension apparatus; a component for applying controlled mechanical loads on the cultivated tissue, wherein each apparatus comprises a pneumatic piston or loading mechanism for applying force directly on the tissue or through a loading mechanism; a component for manual and/or automatic control of the applied load using a computing system wherein the computing system determines, supplies, and controls the voltage input sent to the loading mechanism control unit; sensing devices and apparatus for intermittently, continuously, or sparingly monitoring tissue properties, including height, changes, load, volume, and temperature, wherein the sensing devices include sensors, electrodes, load cells, or spectrophotometers; and a plurality of tubes, ports, and liquid pumps to control the flow of CPA to each compression apparatus.

In any or all of the above embodiments, the compression and/or tension frame system, such as a bioreactor wherein each apparatus is run independently of other compression apparatus.

In any or all of the above embodiments, each apparatus is run simultaneously with other compression and/or tension apparatus.

In any or all of the above embodiments, the computing system is coupled with a data acquisition unit controlling each compression and/or tension apparatus.

In any or all of the above embodiments, the sensing devices and apparatus are coupled to each compression and/or tension apparatus, allowing for monitoring of tissue and solution properties.

In any or all of the above embodiments, the circulation of nutrients is achieved through tubes connected to the compression and/or tension apparatus.

In any or all of the above embodiments, the circulation of nutrients is achieved through the loading mechanism.

### EXAMPLES

### Example 1

In this example, NP cells were isolated following a protocol adapted and modified from Wiseman et al. (Wiseman, M. A., H. L. Birch, M. Akmal, and A. E. Goodship, Spine, 2005, 30:505-11). Two freshly skinned skeletally mature bovine tails were obtained from a local abattoir, disinfected for five minutes in a 1% Betadine solution (RC3955, VWR International, PA), and allowed to dry in a sterile environment for ten minutes. Muscle and fat tissue were removed from each tail, exposing the IVDs. The isolated IVDs were wrapped with a sterile gauze soaked in 0.9% sodium chloride (IC102892.5, VWR International, PA) and 55 mM sodium citrate (BDH9288, VWR International, PA) while awaiting dissection.

NP tissue was removed with a scalpel, cut into 4 mm² pieces, and digested in a humidified cell incubator at 37° C. and 5% CO₂. Digestion involved a one-hour incubation period in DMEM (D6046, Sigma) with 0.2% Pronase E (97062-916, VWR International, PA), centrifugation at 400 RCF for 10 minutes to remove the supernatant, and incubation in Dulbecco Modified Eagle Medium (DMEM) (D6046, Sigma, MO) with 0.025% Collagenase (103701-190, VWR International, PA) for 18 hours. Digested NP tissue was filtered using a 70-μm cell strainer (CLS431751, Sigma, MO) and washed twice with 1×PBS. Cells in the filtrate were also washed twice with 1×PBS, resuspended in cell culture media solution (89% DMEM (D6046, Sigma, MO), 10% FBS (F0926, Sigma, MO), and 1% antibiotic/antimycotic solution (A5955, Sigma, MO), and seeded in monolayer cultures at their appropriate seeding density. Cells were passaged three times prior to performing cell viability assays.

### Example 2

In this example, Confluent NP cells were detached from the flasks using a 0.25% trypsin solution (82003-688, VWR International). Cells were centrifuged at 400 RCF for 10 minutes then resuspended in 2% low-viscosity alginate (A0682, Sigma, MO) in 0.15M NaCl (IC102892.5, VWR International, PA) to a final density of 300,000 cells/mL. The alginate cell solution was slowly dropped into 0.15M CaCl₂ (C4901, Sigma, MO) using a 16G needle, incubated for 15 minutes on a rocker, washed twice with 0.15M NaCl, and incubated in cell culture media for 24 hours at 5% CO₂ at 37° C. before performing cryopreservation toxicity test.

### Example 3

In this example, NP cells were cultured in 6 well-plates to elucidate the cytotoxicity of CPA 1 composed of 80% DMEM (D6046, Sigma), 10% DMSO (IC0219605590, VWR International, PA), and % 10 propylene glycol (PG) (P4347, Sigma, MO). The wells were assigned randomly to incubation times of 2, 6, 12, 18, or 24 hours (n=3) at 4° C. Cells were stained with a fluorescent LIVE/DEAD™ assay (L3224, ThermoFisher, MA) and scanned using Keyence BZ-X700 fluorescence microscope (Oregon State University, OR). The captured scans were processed on ImageJ 1.53i to estimate cell viability (Schneider, Rasband, and Eliceiri 2012).

### Example 4

In this example, the interaction between the CPA type (CPA 1 and CPA 2) and incubation time on cell viability at 4° C. were assessed. CPA 2 included 80% DMEM (D6046, Sigma), 10% DMSO (IC0219605590, VWR International, PA), and % 10 ethylene glycol (EG) (324558, Sigma, MO). Five encapsulated beads were assigned randomly to a CPA type and an incubation time of 0, 2, 4, 8, or 48 hours. Cells were stained using the LIVE/DEAD™ assay (L3224, ThermoFisher, MA) and scanned with Keyence BZ-X700. The captured scans were processed on ImageJ 1.53i to estimate cell viability (Schneider, Rasband, and Eliceiri 2012).

### Example 5

In this example, the transport of DMSO in free-swelling IVDs was assessed. Three skeletally mature Black Angus bovine IVDs were incubated in 10% DMSO for three days and CT-scanned at 0, 24, 56, and 72 hours. DMSO penetration was tracked using a Toshiba Aquilion 64-slice Computed Tomography (CT) unit provided by the Carlson College of Veterinary Medicine at Oregon State University. Acquisition parameters were set to 120 kVp, 240 FoV, 0.6 pitch, and 250 mA.

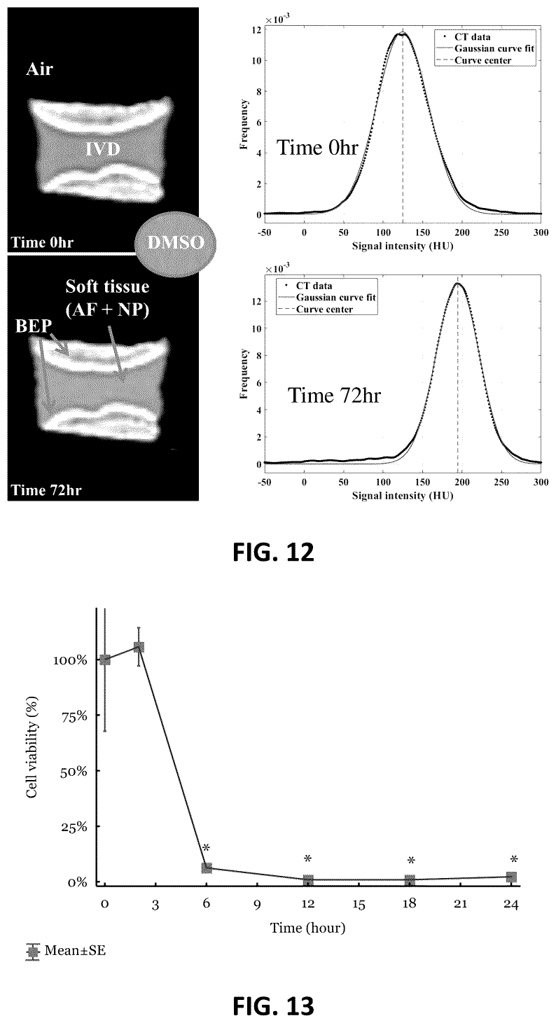

Materialize Mimics® 21.0 was used to segment the IVD's soft tissue (AF and NP). A code was developed in MATLAB® 9.9 to perform a Gaussian curve-fitting analysis of the segmented scans, estimating the mean signal intensity (FIG. 12).

As shown in FIG. 12, the mean signal intensity of the IVD's soft tissue increased due to the penetration of DMSO. CT scans of IVDs were segmented in Materialize Mimics@ 21.0 (left), and a Gaussian curve was fitted in MATLAB® 9.9 (right) to find the mean signal intensity in the tissue.

The correlation in equation 1 was developed To relate mean signal intensity to penetration %, the mean signal intensity was normalized for the sample of interest (IVDₙ) to the intensities of both a fresh IVD (IVD₀) and a fully-saturated IVD (IVD_{F}*).

$$\frac{\frac{{IVD}_{n}}{{IVD}_{F}} - \frac{{IVD}_{0}}{{IVD}_{F}}}{\frac{{IVD}_{F}}{{IVD}_{F}} - \frac{{IVD}_{0}}{{IVD}_{F}}} \qquad Equation 1$$

IVD_{F} was soaked in 10% DMSO for a week-CT scan showed to further increase in DMSO penetration after a week—data not shown).

The effectiveness of PrimeGrowth™ (n=3) and compression (n=3) in improving the transport of DMSO and decreasing cell CPA exposure time were also assessed. PrimeGrowth™ is designed to clear blood clots in the CEP, improving transport through its pores and channels. Axial compression could enhance the convection transport of fluids in the IVD. IVDs treated with PrimeGrowth™ were incubated in a 30 mL Isolation Medium for one hour and rinsed in an equal volume of Neutralization Medium for two minutes. In a separate protocol, bovine IVDs were compressed in a custom-built bioreactor for four hours under a sine-wave load (0 MPa to 2.5 MPa at 0.1 Hz) and soaked in 10% DMSO. DMSO penetration in IVDs was tracked via CT scans compared to a control group (n=3) soaking in 10% DMSO. CT scans were acquired at 0, 5, 8, 24, and 36 hours.

### Example 6

In this example, IVDs were cryopreserved using our optimized method comprised of compressing the IVDs and then allowing them to free-swell in CPA 2. A total of 16 IVDs were isolated from freshly harvested bovine tails and assigned randomly to four groups: fresh, no C.C. (IVDs were frozen without CPA 2 or compression), CPA (IVDs soaked in CPA 2 without compression prior to cryopreservation), and C.C. (IVDs were compressed then free-soaked in CPA2 for four hours prior to cryopreservation). The IVDs were cryopreserved at −80° C. with a cooling rate of −1° C./minute. After one week of storage at −80° C., the IVDs were thawed in a warm water bath and removed through a step-wise dilution process using compression and free-swelling. The IVDs were compressed for four hours in 1×PBS and allowed to soak under no load for 3-4 hours. This process was repeated four times. To ensure the dilution of DMSO (to at least 1% of its initial value), osmolarity was tracked in the solution with a micro-osmometer (Advanced@ Model 3320, Advanced Instruments, MA). Once the DMSO was removed, all IVDs were cultured in DMEM up to 12 hours, then cut in the sagittal plane to estimate cell viability.

### Example 7

In this example, a cross section of thickness 2 mm was acquired using a surgical blade at the sagittal center of the IVD to perform a fluorescent LIVE/DEAD™ assay (L3224, ThermoFisher, MA). The manufacturer's protocol recommended incubating tissue in the stain for 45 minutes at room temperature and rinsing the tissue with 1×PBS prior to scanning. Next, the slices were scanned using Keyence BZ-X700 fluorescence microscope (Oregon State University, OR) and processed on ImageJ 1.53i to estimate cell viability in the outer AF, inner AF, and NP.

### Example 8

In this example, conventional protocols for cryopreservation of IVD demonstrated the need for optimization of CPA transport to improve cell viability. The viability of NP cells decreased when exposed to CPA 1 by about 95% at six hours of incubation (FIG. 13).

FIG. 13 shows cell viability of an NP cells in a monolayer culture exposed to CPA 1 at 4° C. for 24 hours (n=3). A one-way ANOVA analysis (p<0.01) indicated significant difference in mean cell viability % between time 0 and all times after 6 hours (times 12, 18, and 24 hours). No significant difference was observed in mean cell viability % between time 0 and 3 hours.

### Example 9

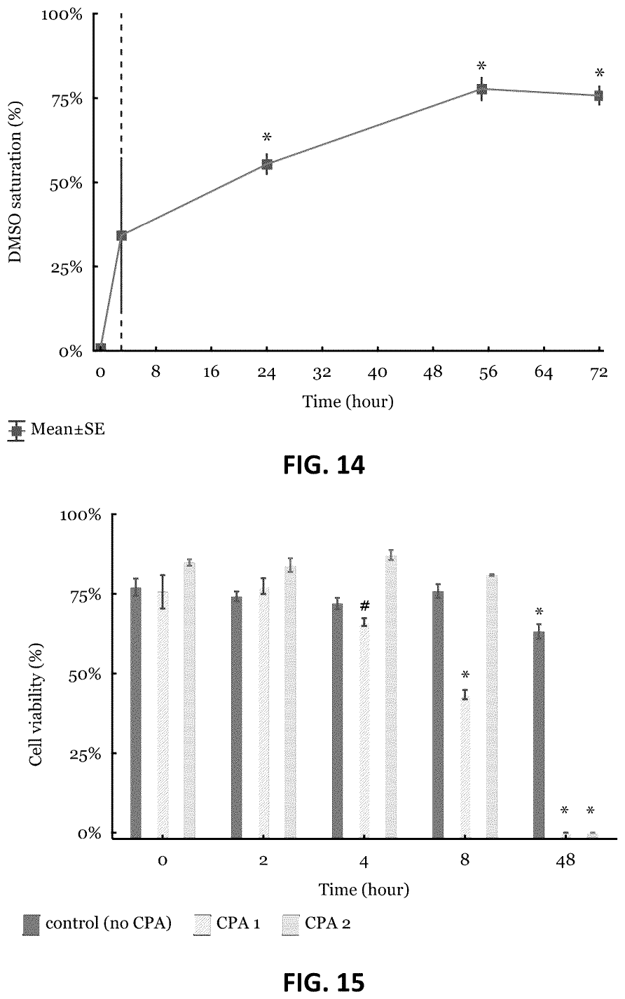

DMSO attenuates CT beam intensity and thus can be used to track its penetration through the IVD's soft tissue. In this example, it was observed in a free swelling IVD that DMSO achieved a 50% saturation level after 24 hours increasing to 75% by 72 hours of incubation. The observed rate of DMSO penetration would lead to total cell death in the IVD before reaching a 100% saturation state (FIG. 14).

FIG. 14 shows average DMSO penetration % in free-swelling IVDs (n=3). Mean DMSO penetration % in the IVD's soft tissue was significantly different after 24 hours of incubation compared to time 0 (one-way ANOVA, p<0.05). The data was normalized to IVDs that had soaked in 10% DMSO for one week. The dashed line shows the time at which significant cell death was observed in the cell culture cytotoxicity experiment (FIG. 13).

### Example 10

Optimization of CPA type required finding a CPA with lower cytotoxic effects compared to PG, which was observed to be devastating for cell viability in a monolayer cell culture (FIG. 13). CPA 2, which replaced PG with EG, showed to decrease cytotoxicity and improve overall cell viability for 8 hours (FIG. 15)

FIG. 15 shows the effect of CPA type and incubation time on the average NP viability encapsulated in alginate. Cells exposed to CPA 1 observed a decreasing viability at 4 hours compared to the control at time 0. CPA 2 was not observed to affect cell viability at 8 hours of incubation. At 48 hours, cell viability was estimated to be 0% for both CPA groups. Significance was estimated by comparing the samples with the control at time 0 with #indicating p<0.05 and * indicating p<0.001 (Factorial ANOVA).

### Example 11

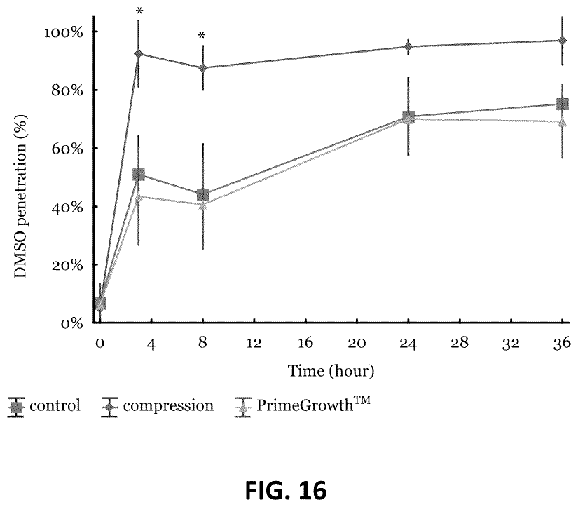

In this example, the rate at which DMSO penetrated through the IVD was still slow when compared to the rate at which cell viability was lost. It required 72 hours for DMSO to reach a 75% level of penetration, while most cells stayed viable only until 8 hours of incubation time. Compression and PrimeGrowth™ were hypothesized to decrease the overall time it required DMSO to fully saturate the IVD's tissue. The results demonstrated compression to be superior to PrimeGrowth™, decreasing the time it required DMSO to fully saturate the disc to 3 hours, a 96% improvement (FIG. 16). In contrast, PrimeGrowth™ showed no detectable improvement to DMSO transport (FIG. 16).

FIG. 16 shows optimization of DMSO penetration % in IVDs (n=3). Compression increased DMSO penetration to 97% compared to the control at 3 hours. IVDs treated with PrimeGrowth™ did not observe improved transport when compared to the control at each time point (Factorial ANOVA, p<0.05).

### Example 12

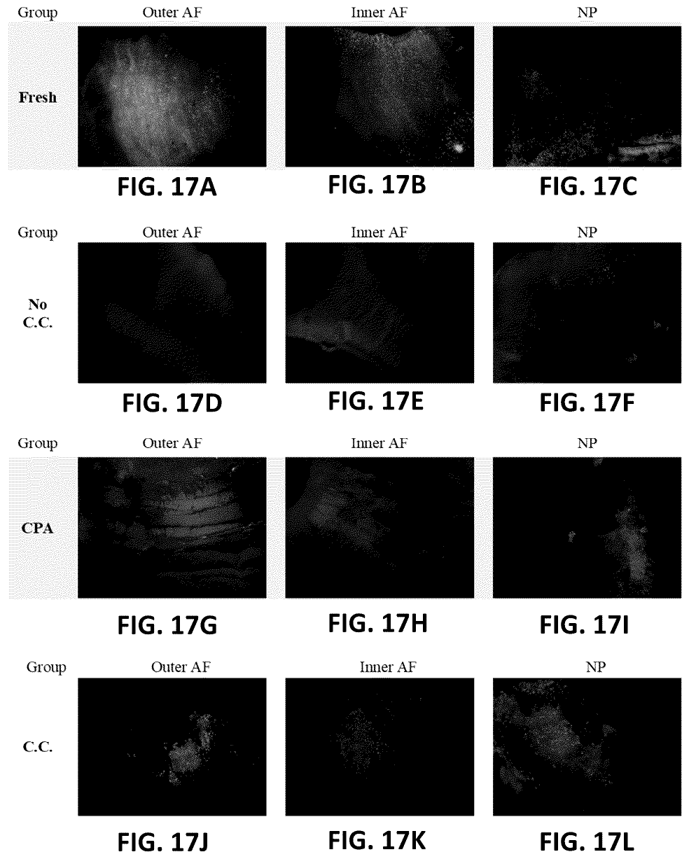

Scans of LIVE/DEAD™ assay from frozen compressed IVDs in CPA 2 showed a significant difference in the number of viable cells and their distribution in the tissue. Cell death in the areas that were sliced by the scalpel were also observed (FIGS. 17A-17L).

FIGS. 17A-17L show representative samples of cell viability fluorescent scans.

### Example 13

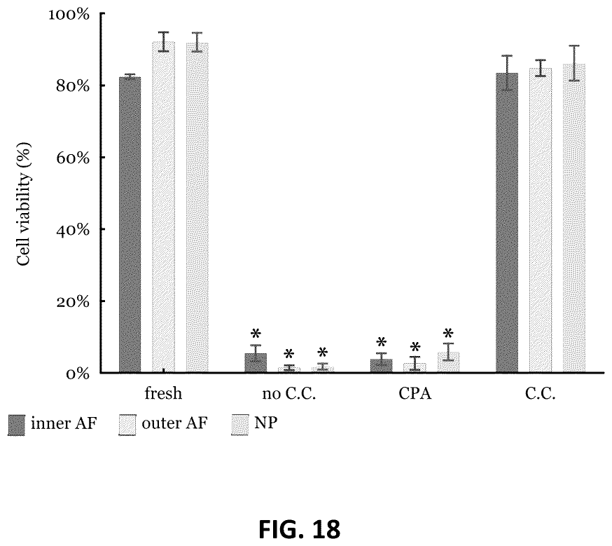

Cell viability was measured in the sagittal plane for all cryopreserved IVDs and compared with a fresh sample. IVDs in treatment groups no C.C. and CPA only showed a ˜5% mean viable cells. IVDs in C.C. group, which were compressed in a custom-built bioreactor to improve transport of CPA 2, had an average of 80-85% viable cells (FIG. 18), which was not significantly different from the fresh unfrozen controls.

FIG. 18 shows mean cell viability in cryopreserved samples (n=4) was improved with compression to speed up transport of DMSO into the IVD. Prior to cryopreservation, IVDs were treated without compression or CPA 2 (no C.C.), with CPA 2 only (CPA), and with compression and CPA 2 (C.C.). Mean cell viability was estimated from sagittal slices and compared against the slices taken from the fresh samples. Significance was denoted by an “*” comparing treatment groups with the fresh control (factorial ANOVA, p<0.05).

These examples show a method to cryopreserve intact bovine IVDs maintaining up to 80% cell viability in all tissue compartments: the outer AF, inner AF, and NP (FIG. 18).

In these examples, the results demonstrated complete cell death after six hours in the monolayer culture and poor cell viability in the alginate beads at eight hours. FIG. 15 shows better cell viability numbers when DMSO was mixed with EG compared to PG (FIG. 15).

X-ray computer tomography (CT) was adopted to track DMSO permeation in the IVD. A slow permeation rate reaching an equilibrium with the surrounding solution after 72 hours was observed (FIG. 14). These results suggested that in these examples commonly practiced incubation times ranging between 2 to 20 hours were insufficient for CPA to fully penetrate the disc and prevent cryoinjury.

Two methods to improve transport in the IVD by (1) clearing transport pathways in the CEPs using PrimeGrowth™ and (2) capitalizing on convective transport through compression were compared. PrimeGrowth™ is a proprietary solution that dissolves blood clots in the porous CEP post IVD harvest, improving solute transport (Grant et al. 2016). The results showed that in these examples the PrimeGrowth™ treatment failed to improve DMSO permeation (FIG. 16). Compressing the IVD provided superior outcomes, decreasing the required transport time by 95% on average to achieve full penetration (FIG. 16). With this technique, the viscoelastic properties of the IVD were utilized. Compression works by decreasing the IVD's height, pushing out the water and other solutes, and decreasing the internal pressure. When the IVD free-swells, it retracts, forcing the dissolved DMSO to flow into the tissue at a faster rate. With this method, about 90% DMSO penetration in 3 to 4 hours was accomplished, preventing cytotoxic and cryogenic injury (FIG. 17).

These examples showed improved cell viability in cryopreserved bovine IVDs and described a method to improve IVD cryopreservation through compression. Bovine IVDs were cryopreserved in 10% DMSO-10% ethylene glycol for over one week. Cell viability was measured in the sagittal plane averaging 80%-85% in the outer AF, inner AF, and NP.

In view of the many possible embodiments to which the principles of the present disclosure may be applied, it should be recognized that the illustrated embodiments are only preferred examples of the disclosure and should not be taken as limiting the scope of the present disclosure. Rather, the scope is defined by the following claims. We therefore claim as our invention all that comes within the scope and spirit of these claims.

We claim:

1. A loading apparatus, comprising:
a loading mechanism for applying tension or compression to a biological material that becomes associated with the loading apparatus during use, the loading mechanism being manually or automatically operable; and
an enclosure constructed with inert materials that contains the loading apparatus and that has a volume to accept a solution comprising a cryopreservation agent, therapeutic agent, restoration agent, or a combination thereof.

2. The loading apparatus of claim 1, wherein:
(i) the loading mechanism comprises a pair of opposing and moveable substrates that are arranged so as to position and hold the biological material between same-facing surfaces of the opposing substrates;
(ii) the loading mechanism comprises an attachment feature that secures the biological material in a fixed position and a compression mechanism that compresses the biological material; or
(iii) the loading mechanism comprises opposing and moveable arms that are capable of holding the biological material and applying tension thereto.

3. The loading apparatus of claim 2, wherein the loading mechanism further comprises a locking mechanism to maintain a compression pressure or tension applied to the biological material.

4. The loading apparatus of claim 1, wherein the loading mechanism is a pneumatic piston or a tensioner.

5. The loading apparatus of claim 1, wherein the enclosure is a bioreactor and further comprises an integration feature that facilitates physical coupling with a cryopreservation storage unit.

6. The loading apparatus of claim 1, further comprising one or more measurement features selected from a displacement sensor, a temperature sensor, a load sensor, or any combination thereof and wherein the one or more measurement features monitors parameters selected from temperature; concentration of the cryopreservation agent, therapeutic agent, restoration agent, or the combination thereof; tension; compression; and/or biological material dimensions during use of the loading apparatus.

7. The loading apparatus of claim 1, wherein the enclosure comprises one or more ports that can be fluidly coupled to one or more tubes and/or liquid pumps that facilitate introducing the cryopreservation agent, therapeutic agent, restoration agent, or the combination thereof into and/or out of the enclosure.

8. The loading apparatus of claim 1, wherein the inert material is a non-ferromagnetic material that avoids generating artifacts during use of the loading apparatus when the loading apparatus is used in combination with one or more medical imaging techniques.

9. A system, comprising:
the loading apparatus of claim 1; and
a controller system in electrical communication with the loading apparatus, wherein the controller system regulates a loading cycle applied to the biological material.

10. The system of claim 9, further comprising:
(i) one or more measurement features that monitors parameters selected from temperature, concentration, tension, compression, and/or biological material dimensions during use of the system;
(ii) one or more imaging data modalities that provides magnetic resonance imaging (MRI) data, computed tomography (CT) data, positron emission tomography (PET) data, ultrasound data, X-ray data, optical scanning data, microwave imaging data, radar ultrasound data, photogrammetry data, elastography data, stereo-imaging data, or a combination thereof for evaluating distribution and attenuation of the cryopreservation agent, therapeutic agent, restoration agent, or the combination thereof within the biological material;
(iii) one or more additional loading apparatuses and one or more additional enclosures, wherein each of the one or more additional loading apparatuses independently is contained within one of the one or more additional enclosures;
(iv) a regulation component for regulating temperature, a CO₂ level, an O₂ level, and/or circulation of nutrients within the enclosure and the one or more additional enclosures;
(v) one or more sensing devices for monitoring biological material properties selected from tensile changes, height changes, volume, and/or temperature, wherein the one or more sensing devices is physically associated with or coupled to the biological material, the loading apparatus (and/or the one or more additional loading apparatus), the enclosure (and/or the one or more additional enclosures), or a combination thereof;
(vi) a plurality of tubes, ports, and liquid pumps that are independently physically or fluidly coupled to one or more of the enclosure (and/or the one or more additional enclosures) and that facilitate delivery of the cryopreservation agent, therapeutic agent, restoration agent, or the combination thereof into and/or out of the system;
(vii) a data acquisition unit that is electronically coupled to the system; or
(viii) any combination of two or more of (i)-(vii).

11. The system of claim 9, wherein the controller system is electronically coupled with the one or more sensing devices and/or the loading mechanism of the loading apparatus, and wherein the controller system is operable to control system parameters and facilitate real-time adjustments during use of the system.

12. A method, comprising:
exposing a biological material to a cryopreservation agent, therapeutic agent, restoration agent, or a combination thereof;
applying a load to the biological material using a loading apparatus; and
removing the load from the biological material;
wherein applying or removing the load facilitates distribution of the cryopreservation agent, therapeutic agent, restoration agent, or the combination thereof within the biological material.

13. The method of claim 12, wherein applying the load to the biological material comprises compressing the biological material using the loading apparatus and removing the load facilitates distribution of the cryopreservation agent, therapeutic agent, restoration agent, or the combination thereof within the biological material by allowing the biological material to decompress back to its natural state.

14. The method of claim 12, wherein applying the load facilitates distribution of the cryopreservation agent, therapeutic agent, restoration agent, or the combination thereof within the biological material by stretching the biological material and removing the load comprises releasing the biological material from the stretched position.

15. The method of claim 12, wherein the cryopreservation agent is selected from dimethylsulfoxide (DMSO), ethylene glycol, propylene glycol, butanediol, cyclohexanediol, diethylene glycol, 3-methoxy-1,2 propanediol, glycerol, formamide, acetamide, propionamide, lactamide, malonamide, dimethylformamide, methylacetamide, dimethylacetamide, betaine, monoacetin, triacetin, ice recrystallization inhibitors, antioxidants, apoptosis inhibitors, metformin, alginate, egg yolk, polyethylene glycol (PEG), polyvinylpyrrolidone (PVP), raffinose, sucrose, trehalose, ethanol, ficoll, polyvinyl alcohol (PVA), methylsulfonylmethane (MSM), polyethyleneimine (PEI), hydroxypropyl cellulose, dextran, or combinations thereof.

16. The method of claim 12, wherein the therapeutic agent may be selected from anti-inflammatory agents, immunosuppressive agents, immunomodulatory agents, vaccines, chondrogenic differentiation agents, cancer-specific inhibitors, gene therapies, anti-aging, anti-fibrotic agents, cell-survival signaling agents, pain management agents, osteogenesis agents, pan-caspase inhibitors, senolytics/senotherapeutics, antiapoptotic agents, or antibiotic drugs; and/or the restoration agent can be a nutrient.

17. The method of claim 12, wherein the biological material is selected from living tissue, donor tissue, tissue composites, scaffolds, artificially manufactured tissues, biocompatible materials, or combinations thereof.

18. The method of claim 12, further comprising obtaining a signal and quantifying an amount of the cryopreservation agent, therapeutic agent, restoration agent, or combination thereof that has permeated the biological material using one or more imaging data modalities selected from MRI data, CT data, PET data, ultrasound data, X-ray data, optical scanning data, microwave imaging data, radar ultrasound data, photogrammetry data, elastography data, or stereo-imaging data.

19. The method of claim 12, wherein the cryopreservation agent is used and the method further comprises:
(i) cooling the biological material to a predetermined temperature to provide a cryopreserved biological material;
(ii) storing the cryopreserved biological material in a cryogenic unit that maintains the cryopreserved biological material at a cryopreservation temperature; or
(iii) a combination of (i) and (ii).

20. The method of claim 19, wherein the method further comprises:
(i) thawing the cryopreserved biological material to provide a thawed biological material;
(ii) exposing the thawed biological material to a restoration solution comprising a restoration agent;
(iii) applying a load to the thawed biological material in the presence of the restoration solution using a loading apparatus; and
(iv) removing the load from the thawed biological material;
wherein applying or removing the load facilitates removing the cryopreservation agent from the thawed biological material and also facilitates uniform distribution of the restoration solution within the thawed biological material.

21. The method of claim 20, wherein the biological material is an intervertebral disc.

22. The method of claim 12, wherein the method uses the cryopreservation agent and the method facilitates an increase in an amount of the cryopreservation agent that is distributed throughout the biological material within a time period where minimal to no cell death occurs in the biological material.
